(19) United States
(12) Patent Application Publication
(10) Pub. No.: US 2024/0108639 A1
(43) Pub. Date: Apr. 4, 2024

(54) COMPOSITIONS AND METHODS OF MAKING COCRYSTALS USING DIELECTRIC HEATING WITH DISPERSIVE AND DISTRIBUTIVE MIXING

(71) Applicant: BOARD OF REGENTS, THE UNIVERSITY OF TEXAS SYSTEM, Austin, TX (US)

(72) Inventors: Mohammed MANIRUZZAMAN, Pflugerville, TX (US); Jiaxiang ZHANG, Pflugerville, TX (US)

(21) Appl. No.: 18/256,897

(22) PCT Filed: Dec. 10, 2021
(86) PCT No.: PCT/US2021/062768

§ 371 Date: Jun. 9, 2023

Related U.S. Application Data
(60) Provisional application No. 63/124,659, filed on Dec. 11, 2020.

(51) Int. Cl. A61K 31/616; A61K 9/14; A61K 9/16; A61K 31/192; A61K 31/405; A61K 31/4422; A61K 31/496; A61K 31/55

(52) U.S. Cl. CPC A61K 31/616; A61K 9/145; A61K 9/1694; A61K 31/192; A61K 31/405; A61K 31/4422; A61K 31/496; A61K 31/55

(57) ABSTRACT

The present disclosure provides methods of preparing pharmaceutical compositions containing cocrystals through the combination application of dielectric heating and distributive and dispersive mixing such as hot melt extrusion (HME). The cocrystals used in these compositions may be formed using an active pharmaceutical ingredient and a co-former. The co-former may be either an excipient or a second active pharmaceutical ingredient. These pharmaceutical compositions may be used in the treatment of a disease or disorder.

This application claims the benefit of priority to U.S. Provisional Application No. 63/124,659, filed on Dec. 11, 2020, the entire contents of which are hereby incorporated by reference.

### BACKGROUND

### 1. Field

The present disclosure relates generally to the field of pharmaceuticals and pharmaceutical manufacture. More particularly, it concerns compositions and methods of preparing a pharmaceutical cocrystal and dosage forms thereof using a combination application of dielectric heating and distributive and dispersive mixing such as hot melt extrusion (HME).

### 2. Description of Related Art

Nowadays, the poor aqueous solubility of APIs always is an obstacle to the current pharmaceutical product development and drug's bioavailability. There are many reports and statistics showed that around 40% of active new chemical entities (NCEs) can be classified as poorly water soluble or insoluble (Heimbach et al., 2007). In order to improve the bioavailability of such APIs, researchers have to develop suitable formulation compositions for such drugs according to the complex but independently acted factors including the biological, physiochemical, physiological, and anatomical characteristics of materials and/or patients. Several approaches have been tried to increase the dissolution rate of poorly water soluble drug i.e. by increasing apparent equilibrium drug solubility or facilitating a formation of metastable supersaturation of drug solutions, such as nanosuspension (Agrawal et al., 2011), hydrotrophy (Maheshwari et al., 2007), salt formation (Paulekuhn, et al., 2007; Elder et al., 2013), amorphous solid dispersions (Crowley et al., 2007; Zhang et al., 2016), solubilization in surfactant micelles (Rosen and Kunjappu, 2012; Vinarov et al., 2018), or lipid-based formulations (Pouton, 2006; Gao and Morozowich, 2006).

The cocrystallization could be one of the optimal approaches for poorly water soluble pharmaceutical compounds drug delivery systems (DDS) development. Pharmaceutical cocrystal refers to “solids that are crystalline single-phase materials composed of two or more different crystalline compounds generally in a stoichiometric ratio which are neither solvates nor simple salts.” The cocrystal can improve the solubility of the starting material that can enhance the delivery and clinical performance of drug products by modulating drug solubility, pharmacokinetics, and bioavailability. Multidrug could be loaded to cocrystals for the additive or synergistic treatment as well. Also, other than the mechanical properties and solubility enhancement as well as taste masking, cocrystals can be highly patentable as novel drug product intermediates. Various processing technologies including spray drying (SD), hot-melt extrusion (HME), freeze-drying (FD), rotary evaporation (RE), cogrinding, spin-coated film (SCF), centrifuge vacuum drying (CVD), supercritical fluid technology, electrostatic spinning have been used to produce cocrystals. But most of the reported studies have so far utilized various technologies such as 1) solution-based methods (e.g. solvent evaporation (Manin et al., 2014), slurry crystallization (Abourahma et al., 2011)), 2) grinding methods (Karki et al., 2007) and 3) melt methods (hot melt extrusion) for the development of pharmaceutical cocrystals. Unfortunately, none, except for HME with some limited extent, of the reported technologies are scalable for large scale commercial manufacturing of pharmaceutical cocrystals. For an example, HME has been used to develop pharmaceutical cocrystals as a potential continuous manufacturing technology but the scale at which successful cocrystals were formulated were in grams rather than in kilograms.

Therefore, there remains a need to develop new methods of preparing cocrystals that may be used to produce cocrystals on a commercial scale.

### SUMMARY

The present disclosure provides methods of preparing pharmaceutical compositions comprising one or more cocrystals using dielectric heating—with a dispersive and distributive mixing process such as a hot melt extrusion process.

In some aspects, the present disclosure provides methods of preparing a pharmaceutical composition comprising:
(A) obtaining a mixture of an active pharmaceutical ingredient (API) and a co-former;
(B) subjecting the mixture to dielectric heating to obtain a pharmaceutical composition;
wherein the pharmaceutical composition comprises at least 50% of the API and the co-former is present as a co-crystal.

In some embodiments, at least 80% of the API and the co-former is present as a co-crystal. In some embodiments, at least 90% of the API and the co-former is present as a co-crystal. In some embodiments, at least 95% of the API and the co-former is present as a co-crystal. In some embodiments, at least 98% of the API and the co-former is present as a co-crystal. In some embodiments, at least 99% of the API and the co-former is present as a co-crystal.

In some embodiments, the dielectric heating comprises using a specific frequency electromagnetic radiation. In some embodiments, the specific frequency electromagnetic radiation is a radio wave. In some embodiments, the radio wave has a frequency from about 10 MHz to about 20 MHz. In other embodiments, the specific frequency electromagnetic radiation is a microwave. In some embodiments, the microwave has a frequency greater than 100 MHz. In some embodiments, the microwave has a frequency from about 500 MHz to about 1,000 GHz. In some embodiments, a frequency from about 1000 MHz to about 100 GHz. In some embodiments, a frequency from about 1000 MHz to about 25 GHz. In some embodiments, the microwave has a frequency from about 1000 MHz to about 10 GHz. In some embodiments, the microwave has a frequency from about 1000 MHz to about 3000 MHz.

In some embodiments, the dielectric heating comprise a heating power. In some embodiments, the heating power is from about 200 W to about 10 kW. In some embodiments, the heating power is from about 500 W to about 5 kW. In some embodiments, the heating power is from about 750 W to about 2 kW. In some embodiments, the heating power is from about 800 W to about 1,500 W. In some embodiments, the dielectric heating comprises using energy having a specific wavelength. In some embodiments, the specific wavelength is greater than 1 mm. In some embodiments, the specific wavelength is from about 1 mm to about 1 m. In some embodiments, the specific wavelength is from about 3 mm to about 300 mm. In some embodiments, the specific wavelength is from about 50 mm to about 200 mm. In some embodiments, the specific wavelength is from about 100 mm to about 150 mm.

In some embodiments, the method further comprising subjecting the mixture to a composition processing method. In some embodiments, the composition processing method is performed contemporaneously with subjecting the mixture to dielectric heating. In some embodiments, the composition processing method is a method which results in a dispersive and distributive mixing process. In some embodiments, the composition processing method is performed after with subjecting the mixture to dielectric heating. In some embodiments, the composition processing method is performed before with subjecting the mixture to dielectric heating.

In some embodiments, the composition processing method is extrusion, fluidized bed granulation, high shear granulation, propeller mixing, turbine mixing, high shear mixing, high pressure or ultrasonic homogenization. In some embodiments, the composition processing method is extrusion. In some embodiments, the extrusion is hot melt extrusion. In some embodiments, the extrusion comprises heating the extrusion composition to a first temperature. In some embodiments, the first temperature is from ambient temperature to a temperature less than the melting of either the co-former or the API. In some embodiments, the first temperature is from about 10° C. to about 250° C. In some embodiments, the first temperature is from about 50° C. to about 150° C.

In some embodiments, the methods comprise a second temperature. In some embodiments, the second temperature is from about 10° C. to about 250° C. In some embodiments, the second temperature is from about 10° C. to about 100° C. In some embodiments, the extrusion method comprises a screw speed from about 10 rpm to about 400 rpm. In some embodiments, the screw speed is form about 20 rpm to about 300 rpm. In some embodiments, the screw speed is from about 25 rpm to about 200 rpm. In some embodiments, the screw speed is 50 rpm, 75 rpm, 100 rpm, 150 rpm, or 200 rpm.

In some embodiments, the productivity or throughput of extrusion is about 100 g/hr to 2.5 kg/hr relative to a lab scale twin-screw extruder. In some embodiments, the productivity of extrusion is about 250 g/hr to 2.0 kg/hr. In some embodiments, the productivity of extrusion is about 360 g/hr, 500 g/hr, 540 kg/hr, 1.08 kg/hr, 2.0 kg/hr, or 2.5 kg/hr.

In some embodiments, the API is a BCS Class II drug. In some embodiments, the API is a BCS Class IV drug. In some embodiments, the API is an API with a melting point of less than 250° C. In some embodiments, the melting point is less than 200° C. In some embodiments, the API is selected from anticancer agents, antiallergic agents, antifungal agents, psychiatric agents such as analgesics, consciousness level-altering agents such as anesthetic agents or hypnotics, nonsteroidal anti-inflammatory agents (NSAIDs), anthelmintics, antiacne agents, antianginal agents, antiarrhythmic agents, anti-asthma agents, antibacterial agents, anti-benign prostate hypertrophy agents, anticoagulants, antidepressants, antidiabetics, antiemetics, antiepileptics, antigout agents, antihypertensive agents, anti-inflammatory agents, antimalarials, antimigraine agents, antimuscarinic agents, antineoplastic agents, anti-obesity agents, antiosteoporosis agents, antiparkinsonian agents, antiproliferative agents, antiprotozoal agents, antithyroid agents, antitussive agent, anti-urinary incontinence agents, antiviral agents, anxiolytic agents, appetite suppressants, beta-blockers, cardiac inotropic agents, chemotherapeutic drugs, cognition enhancers, contraceptives, corticosteroids, Cox-2 inhibitors, diuretics, erectile dysfunction improvement agents, expectorants, gastrointestinal agents, histamine receptor antagonists, immunosuppressants, keratolytics, lipid regulating agents, leukotriene inhibitors, macrolides, muscle relaxants, neuroleptics, nutritional agents, opioid analgesics, protease inhibitors, and sedatives. In some embodiments, the API is an antifungal agent, a psychiatric agent, an antiallergic agent, a chemotherapeutic drug, an antibiotic, or a nonsteroidal anti-inflammatory agent. In some embodiments, the API is a chemotherapeutic drug. In other embodiments, the API is an antibiotic. In other embodiments, the API is a nonsteroidal anti-inflammatory agent such as ibuprofen or acetylsalicylic acid. In other embodiments, the API is an antihypertensive agent such as nifedipine. In other embodiments, the API is an antifungal agent such as indomethacin. In other embodiments, the API is an antiepileptic such as carbamazepine. In some embodiments, the API is a psychiatric agent such as aripiprazole. In some embodiments, the API is an antiallergic agent such as tranilast.

In some embodiments, the co-former interacts with the API through one or more non-covalent interactions. In some embodiments, the non-covalent interactions are ionic interactions, hydrogen bonding, halogen bonding, van der Waals forces, π-π interactions, or hydrophobic effects. In some embodiments, the co-former and the API interact with two or more non-covalent interactions. In some embodiments, the co-former is a compound which modifies the solubility of the API. In some embodiments, the co-former is a compound which is sparingly soluble and modifies the solubility of the API. In some embodiments, the co-former is a compound which is sensitive to the environment and modifies the solubility of the active pharmaceutical ingredient. In some embodiments, the compound is sensitive to the pH of the environment. In some embodiments, the compound is sensitive to the temperature of the environment. In some embodiments, the co-former is a compound that has no therapeutic effect. In some embodiments, the co-former is a second API. In some embodiments, the second API is for the same disease or disorder as the first API. In some embodiments, the second API is for a different disease or disorder as the first API.

In some embodiments, the co-former comprises one or more functional groups selected from amine, amide, a nitrogen containing heterocycle, carbonyl, carboxyl, hydroxyl, phenol, sulfone, sulfine, sulfinyl, sulfonyl, mercapto, and methyl thio. In some embodiments, the functional group is a NH₂, OH, C(O), C(O)OH, SH, or a nitrogen containing heterocycle. In some embodiments, the functional group is a nitrogen containing heterocycle, NH₂, OH, or SH. In some embodiments, the co-former is a carboxylic acid such as malic acid. In other embodiments, the co-former is a vitamin or a vitamin derivative such as nicotinamide. In other embodiments, the co-former is a flavoring agent such as saccharin.

In some embodiments, the pKₐ of the active pharmaceutical ingredient and the pKₐ of the co-former have a pKₐ difference of less than 3. In some embodiments, the pKₐ difference is less than 2. In some embodiments, the pKₐ difference is less than 1. In some embodiments, the pKₐ difference is less than 0.5.

In some embodiments, the methods result in a compositions showing improved flowability or is able to obtain more co-crystals in the pharmaceutical composition relative to either dielectric heating or an extrusion process alone. In some embodiments, the mixture further comprises an excipient.

In some embodiments, the methods further comprise one or more further formulation steps. In some embodiments, the further formulation steps including milling or grinding. In some embodiments, the further formulation steps comprise tableting, filling a capsule, formulating an oral suspension, formulating a film, or additive manufacturing techniques. In some embodiments, the additive manufacturing technique is vat photopolymerization, material jetting, binding jetting, powder-bed fusion, material extrusion, directed energy deposition, sheet lamination, fused deposition modeling, binder spraying, or selective laser sintering.

In still another aspect, a pharmaceutical compositions comprising:
(A) an active pharmaceutical ingredient (API);
(B) a co-former;
   wherein at least 50% of the API and the co-former is present as a co-crystal; and the pharmaceutical composition has been subjected to dielectric heating.

In some embodiments, at least 80% of the API and the co-former is present as a co-crystal. In some embodiments, at least 90% of the API and the co-former is present as a co-crystal. In some embodiments, at least 95% of the API and the co-former is present as a co-crystal. In some embodiments, at least 98% of the API and the co-former is present as a co-crystal. In some embodiments, at least 99% of the API and the co-former is present as a co-crystal. In some embodiments, the co-crystals are in a single phase. In some embodiments, the pharmaceutical compositions comprise a Carr's Index from about 5 to about 30. In some embodiments, the pharmaceutical compositions comprise a surface area of greater than 100 m²/g. In some embodiments, the pharmaceutical compositions comprise a mean or average particle size distribution is from about 25 μm to about 500 μm. In some embodiments, the mean or average particle size distribution is from about 50 μm to about 250 μm. In some embodiments, the pharmaceutical compositions have a flowability as a function of angle of repose of greater than about 25. In some embodiments, the pharmaceutical composition comprises a drug content uniformity is from about 95% to about 105%.

In some embodiments, the dielectric heating comprises using a specific frequency electromagnetic radiation. In some embodiments, the specific frequency electromagnetic radiation is a radio wave. In some embodiments, the radio wave has a frequency from about 10 MHz to about 20 MHz. In other embodiments, the specific frequency electromagnetic radiation is a microwave. In some embodiments, the microwave has a frequency greater than 100 MHz. In some embodiments, the microwave has a frequency from about 500 MHz to about 1,000 GHz. In some embodiments, a frequency from about 1000 MHz to about 100 GHz. In some embodiments, a frequency from about 1000 MHz to about 25 GHz. In some embodiments, the microwave has a frequency from about 1000 MHz to about 10 GHz. In some embodiments, the microwave has a frequency from about 1000 MHz to about 3000 MHz.

In some embodiments, the dielectric heating comprise a heating power. In some embodiments, the heating power is from about 200 W to about 10 kW. In some embodiments, the heating power is from about 500 W to about 5 kW. In some embodiments, the heating power is from about 750 W to about 2 kW. In some embodiments, the heating power is from about 800 W to about 1,500 W. In some embodiments, the dielectric heating comprises using energy having a specific wavelength. In some embodiments, the specific wavelength is greater than 1 mm. In some embodiments, the specific wavelength is from about 1 mm to about 1 m. In some embodiments, the specific wavelength is from about 3 mm to about 300 mm. In some embodiments, the specific wavelength is from about 50 mm to about 200 mm. In some embodiments, the specific wavelength is from about 100 mm to about 150 mm.

In some embodiments, the API is a BCS Class II drug. In some embodiments, the API is a BCS Class IV drug. In some embodiments, the API is an API with a melting point of less than 250° C. In some embodiments, the melting point is less than 200° C. In some embodiments, the API is selected from anticancer agents, antiallergic agents, antifungal agents, psychiatric agents such as analgesics, consciousness level-altering agents such as anesthetic agents or hypnotics, nonsteroidal anti-inflammatory agents (NSAIDs), anthelmintics, antiacne agents, antianginal agents, antiarrhythmic agents, anti-asthma agents, antibacterial agents, anti-benign prostate hypertrophy agents, anticoagulants, antidepressants, antidiabetics, antiemetics, antiepileptics, antigout agents, antihypertensive agents, anti-inflammatory agents, antimalarials, antimigraine agents, antimuscarinic agents, antineoplastic agents, anti-obesity agents, antiosteoporosis agents, antiparkinsonian agents, antiproliferative agents, antiprotozoal agents, antithyroid agents, antitussive agent, anti-urinary incontinence agents, antiviral agents, anxiolytic agents, appetite suppressants, beta-blockers, cardiac inotropic agents, chemotherapeutic drugs, cognition enhancers, contraceptives, corticosteroids, Cox-2 inhibitors, diuretics, erectile dysfunction improvement agents, expectorants, gastrointestinal agents, histamine receptor antagonists, immunosuppressants, keratolytics, lipid regulating agents, leukotriene inhibitors, macrolides, muscle relaxants, neuroleptics, nutritional agents, opioid analgesics, protease inhibitors, and sedatives. In some embodiments, the API is a chemotherapeutic drug, a psychiatric agent, an antiallergic agent, an antibiotic, an antihypertensive agent, an antifungal agent, an antiepileptic, or a nonsteroidal anti-inflammatory agent. In some embodiments, the API is a chemotherapeutic drug. In other embodiments, the API is an antibiotic. In other embodiments, the API is a nonsteroidal anti-inflammatory agent such as ibuprofen or acetylsalicylic acid. In other embodiments, the API is an antihypertensive agent such as nifedipine. In other embodiments, the API is an antifungal agent such as indomethacin. In other embodiments, the API is an antiepileptic such as carbamazepine. In some embodiments, the API is a psychiatric agent such as aripiprazole. In some embodiments, the API is an antiallergic agent such as tranilast.

In some embodiments, the co-former interacts with the API through one or more non-covalent interactions. In some embodiments, the non-covalent interactions are ionic interactions, hydrogen bonding, halogen bonding, van der Waals forces, π-π interactions, or hydrophobic effects. In some embodiments, the co-former and the API interact with two or more non-covalent interactions. In some embodiments, the co-former is a compound which modifies the solubility of the API. In some embodiments, the co-former is a compound which is sparingly soluble and modifies the solubility of the API. In some embodiments, the co-former is a compound which is sensitive to the environment and modifies the solubility of the active pharmaceutical ingredient. In some embodiments, the compound is sensitive to the pH of the environment. In some embodiments, the compound is sensitive to the temperature of the environment. In some embodiments, the co-former is a compound that has no therapeutic effect. In some embodiments, the co-former is a second API. In some embodiments, the second API is for the same disease or disorder as the first API. In some embodiments, the second API is for a different disease or disorder as the first API.

In some embodiments, the co-former comprises one or more functional groups selected from amine, amide, a nitrogen containing heterocycle, carbonyl, carboxyl, hydroxyl, phenol, sulfone, sulfine, sulfinyl, sulfonyl, mercapto, and methyl thio. In some embodiments, the functional group is a NH₂, OH, C(O), C(O)OH, SH, or a nitrogen containing heterocycle. In some embodiments, the functional group is a nitrogen containing heterocycle, NH₂, OH, or SH. In some embodiments, the co-former is a carboxylic acid such as malic acid. In other embodiments, the co-former is a vitamin or a vitamin derivative such as nicotinamide. In other embodiments, the co-former is a flavoring agent such as saccharin.

In some embodiments, the pKₐ of the active pharmaceutical ingredient and the pKₐ of the co-former have a pKₐ difference of less than 3. In some embodiments, the pKₐ difference is less than 2. In some embodiments, the pKₐ difference is less than 1. In some embodiments, the pKₐ difference is less than 0.5.

In some embodiments, the pharmaceutical compositions further comprise an excipient. In some embodiments, the pharmaceutical compositions are formulated for administration: orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularlly, intravitreally, liposomally, locally, mucosally, parenterally, rectally, subconjunctival, subcutaneously, sublingually, topically, transbuccally, transdermally, vaginally, in crèmes, in lipid compositions, via a catheter, via a lavage, via continuous infusion, via infusion, via inhalation, via injection, via local delivery, or via localized perfusion. In some embodiments, the pharmaceutical composition has been formulated for oral administration. In some embodiments, the pharmaceutical composition is present as a capsule, tablet, oral suspensions, oral films, or chewable dosages. In some embodiments, the API is acetylsalicylic acid, indomethacin, ibuprofen, carbamazepine, or nifedipine and the co-former is nicotinamide or malic acid.

In yet another aspect, the present disclosure provides pharmaceutical compositions prepared according to the methods described herein.

In another aspect, the present disclosure provides methods of treating or preventing a disease or disorder comprising administering a therapeutically effective amount of a pharmaceutical composition described herein or a pharmaceutical composition prepared as described herein, wherein the therapeutically active agent is useful for treating or preventing the disease or disorder.

In yet another aspect, the present disclosure describes compositions comprising:
(A) an active pharmaceutical ingredient (API);
(B) a co-former;
wherein at least 50% of the API and the co-former are present in a substantially liquid phase.

In some embodiments, the composition comprises at least 80% of the API and the co-former are present in a substantially liquid phase. In some embodiments, the composition comprises least 90% of the API and the co-former are present in a substantially liquid phase. In some embodiments, the composition comprise at least 95% of the API and the co-former are present in a substantially liquid phase.

Other objects, features, and advantages of the present disclosure will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-1C shows a typical schematic demonstrates using dielectric technologies to the development of optimized intermediate materials such as cocrystals for a further downstream process and optimized drug products using cocrystals for a patient without further downstream process. FIG. 1C shows a demonstration of the instruments set up used here.

FIG. 2 shows a demonstration of conjugating dielectric heating and HME platforms.

FIG. 3 shows the optimization of oral drug administration via dielectric heating/HME printing technologies.

FIG. 4 shows a demonstration of a typical CoMIDEx process with varying throughput (from −350 g/h to 1.5 kg/h).

FIG. 5 shows the polarized light microscope pictures of the physical mixtures and cocrystals of each formulation.

FIG. 6 shows the DSC curve (left panel) of the IBU-NTM physical mixture (dashed line) and cocrystals (solid line) produced via dielectric heating. The right panel shows reference work showing IBU-NTM cocrystal melting behavior (Yuliandra et al., 2018).

FIG. 7 shows the XRD spectrum of the IBU, NTM, physical mixtures and the cocrystals produced via dielectric heating and dielectric heating-HME.

FIG. 8 shows the FTIR spectrum of the IBU, NTM, physical mixtures and the cocrystals produced via dielectric heating and dielectric heating-HME.

FIG. 9 shows the Raman spectrum of bulk IBU, NTM, physical mixtures and the cocrystals produced via dielectric heating and dielectric heating-HME.

FIG. 10 shows a schematic demonstration of manufacturing the cocrystal using the batch dielectric heating process.

FIG. 11 shows the polarized light microscope pictures of the physical mixtures and cocrystals of each formulation.

FIG. 12 shows the DSC curve of the IBU-NTM physical mixture (dashed line) and cocrystals (solid line) produced via dielectric heating in the left panel. With right panel showing the reference work shown IBU-NTM cocrystal melting behavior (Yuliandra et al., 2018).

FIG. 13 shows the XRD patterns of NDP, MLA, and the and NDP-MLA cocrystals produced via dielectric heating.

FIG. 14 shows a demonstration of the samples heated on slides and in vials.

FIG. 15 shows the demonstration of the screw configuration and temperature profiles used in the Example 3 compositions.

FIG. 16 shows the photographs of the cocrystal granules obtained from the batches processed at 50, 75, and 150 rpm.

FIG. 17 shows the PLM figures of IBU, NTM, physical mixtures, and IBU-NTM cocrystals.

FIG. 18 shows the DSC curves of IBU, NTM, physical mixtures, and IBU-NTM cocrystals.

FIG. 19 shows the PXRD curves of IBU, NTM, physical mixtures, and IBU-NTM cocrystals.

FIG. 20 shows the FTIR spectra of IBU, NTM, physical mixtures, and IBU-NTM cocrystals.

FIG. 21 shows the raman spectra of IBU, NTM, physical mixtures, and IBU-NTM cocrystals.

FIG. 22 shows the PLM figures of CBZ, MLA, physical mixtures, and CBZ-MLA cocrystals.

FIG. 23 shows the DSC curves of CBZ, MLA, physical mixtures, and CBZ-MLA cocrystals.

FIG. 24 shows the PXRD curves of CBZ, MLA, physical mixtures, and CBZ-MLA cocrystals.

FIG. 25 shows the FTIR spectra of CBZ, MLA, physical mixtures, and CBZ-MLA cocrystals.

FIG. 26 shows the raman spectra of CBZ, MLA, physical mixtures, and CBZ-MLA cocrystals.

FIG. 27 shows the PLM figures of TRA, SCH, physical mixtures, and TRA-SCH cocrystals molecules.

FIG. 28 shows the DSC curves of TRA, SCH, physical mixtures, and TRA-SCH cocrystals molecules.

FIG. 29 shows the FTIR spectra of TRA, SCH, physical mixtures, and TRA-SCH cocrystals molecules.

FIG. 30 shows the raman spectra of TRA, SCH, physical mixtures, and TRA-SCH cocrystals molecules.

FIG. 31 shows the PLM figures of APZ, MLA, physical mixtures, and APZ-MLA cocrystals.

FIG. 32 shows the DSC curves of APZ, MLA, physical mixtures, and APZ-MLA cocrystals.

FIG. 33 shows the PXRD curves of APZ, MLA, physical mixtures, and APZ-MLA cocrystals.

FIG. 34 shows the FTIR spectra of TRA, SCH, physical mixtures, and TRA-SCH cocrystals molecules.

FIG. 35 shows the raman spectra of TRA, SCH, physical mixtures, and TRA-SCH cocrystals molecules.

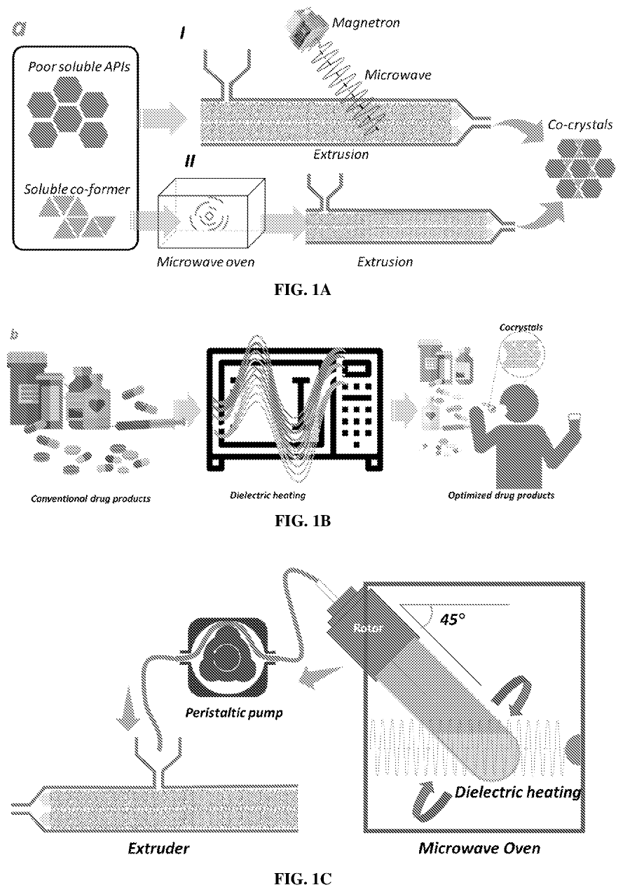

### DESCRIPTION OF ILLUSTRATIVE EMBODIMENTS

The present disclosure relates to the development of the pharmaceutical products composition of cocrystal based formulations by means of dielectric heating techniques coupled with a mixing process such as HME (CoMIDEx). Briefly, the drug delivery system consists of pharmaceutical cocrystals formulated with or without other pharmaceutical excipients and manufactured using customized dielectric heating component such as microwave ovens, microwave reactors, or microwave generator conjugated with a mixing apparatus such as hot melt extrusion.

In order to meet the growing need for scale up and commercial exploitation of pharmaceutical cocrystal manufacturing, a viable continuous microwave induced dielectric heating coupled with a mixing apparatus such as HME technology (CoMIDEx) is described herein. The advantage of this CoMIDEx technology is that it expedites the manufacturing process by the dual effect of dielectric heating and dispersive/distributive mixing during the single-step manufacturing process eliminating all intermediate steps whereas making the process faster. Furthermore, this technology doesn't use any solvent, it is environmentally friendly whilst complying the regulatory expectations.

Conjugating dielectric heating with HME techniques might be an exemplary option in pharmaceutical cocrystals development because of the favorable powder properties generated from this technology, the absence of organic solvents in processing, the small footprint of the equipment, ease of increasing batch size, scalability from pilot to industrial setting, and suitability of continuous processing. These cocrystals based formulations will demonstrate enhanced stability, solubility, and bioavailability with the advantages of on-demand, patient-specific manufacturing. Also, the use of cocrystal offers multidrug loading in the crystalline form and improved physicochemical properties such as flowability, enhanced solubility and other relevant physic-mechanical properties.

### I. Pharmaceutical Compositions

In some aspects, the present disclosure provides methods of using dielectric heating to prepare pharmaceutical compositions containing an active pharmaceutical ingredient or a pharmaceutically acceptable salt, ester, derivative, analog, pro-drug, or solvates thereof and a co-former which may be an excipient or a second active pharmaceutical ingredient as a co-crystal. These compositions may be used to prepare a pharmaceutical composition from a starting material such as a filament or powder that exhibits one or more favorable properties such as exhibiting a free-flowing property as an angle of repose, sufficient strength, sufficient stress, bend angle, diameter, viscosity, or Carr's Index. The co-crystal and the active pharmaceutical ingredient may comprise the active pharmaceutical ingredient and the co-former in a molar ratio from about 0.1 to about 10, from about 0.25 to about 4, or from about 0.5 to about 2. The molar ratio of the active pharmaceutical ingredient and the co-former is from about 0.1, 0.2, 0.25, 0.33, 0.5, 1, 2, 3, 4, 5, or 10. The amount of the composition which contains the co-crystal is greater than about 50%, 60%, 70%, 80%, 90%, 95%, 96%, 97%, 98%, or 99%. The pharmaceutical composition comprises from about 50%, 60%, 65%, 70%, 75%, 80%, 85%, 90%, 92.5%, 95%, 96%, 97%, 97.5%, 98%, 98.5%, 99%, 99.5%, to about 99.9%, or any range derivable therein of the co-crystal. The co-crystals may be formed between an active pharmaceutical ingredient and either an excipient or a second active pharmaceutical ingredient. These components of the co-crystals may have a pKₐ difference of less than 3, less than 2, less than 1.5, less than 1, less than 0.75, less than 0.5, or less than 0.25.

These compositions may exhibit one or more free-flowing properties such as having a flowability as measured by the angle of repose of less than 25. These compositions may exhibit a flowability as measured by the angle of repose of less than about 25, less than about 27.5, less than about 30, less than about 32.5, less than about 35, less than about 37.5, or less than about 40. The flowability may be from about 25 to about 40, or from about 25 to about 30. The flowability may be from about 2, 4, 5, 6, 8, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or any range derivable therein. The flowability of the pharmaceutical composition is measured by, the simplest method for the determination of the angle of repose is the “poured” angle. A funnel with a wide outlet is affixed at a distance of 10 cm above the bench, where a piece of paper is placed directly beneath the funnel. The granules are added while the funnel is closed. The contents flow through and collect on the paper. The diameter of the cone (D) and two opposite sides (l₁+l₂) are measured with rulers. The angle of repose (θ) is calculated from the equation arc cos[D/(l₁+l₂)]. The relationship between flow properties and angle of repose has been established. When the angle of repose is less than 25 degrees, the flow is said to be excellent; on the other hand, if the angle of repose is more than 40 degrees, the flow is considered to be poor. These pharmaceutical compositions may be present as agglomerations and used in either a batch, semi-continuous, continuous manufacturing process. The active pharmaceutical ingredient may act as a binder between the absorbent particles within the pharmaceutical composition.

In other aspects, the present pharmaceutical compositions may exhibit a mean or average particle size distribution greater than 25 μm, greater than 50 μm, or greater than 60 μm. In some embodiments, the pharmaceutical compositions exhibit a mean or average particle size from about 25 μm to about 500 μm, 30 μm to about 400 μm, 35 μm to about 350 μm, 40 μm to about 300 μm, 50 μm to about 250 μm, 50 μm to about 200 μm, 50 μm to about 150 μm, 55 μm to about 125 μm, or from about 60 μm to about 100 μm. The mean or average particle size of the pharmaceutical composition comprises from about 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 105 μm, 110 μm, 115 μm, 120 μm, 125 μm, 150 μm, 175 μm, 200 μm, 250 μm, 300 μm, 350 μm, 400 μm, 450 μm, 500 μm, 600 μm, 700 μm, 800 μm, 900 μm, to about 1000 μm, or any range derivable therein. The mean or average particle size of the pharmaceutical composition may be determined by mesh analysis using a sonic sifter. The particle size distribution of the dried granules can also be determined by a dry laser diffraction technique or scanning electron microscopy.

Similarly, these compositions may exhibit a particle diameter, D₅₀, wherein 50% of the particles in the composition are larger than this particular particle size. The composition may have a D₅₀ of less than 100 μm, less than 75 μm, less than 60 μm, or less than 50 μm. Additionally, the composition may exhibit a particle diameter, D₉₀, wherein 90% of the particles in the composition are smaller than this particular particle size. The particles may have a D₉₀ wherein the D₉₀ is greater than 25 μm, greater than 40 μm, or greater than 50 μm. Alternatively, the D₉₀ may be less than 100 μm, less than 90 μm, less than 80 μm, or less than 75 μm. In some embodiments, the D₉₀ may be from about 10 μm to about 150 μm, from about 25 μm to about 100 μm, from about 50 μm to about 80 μm. The D₉₀ may be from about 10 μm, 25 μm, 30 μm, 40 μm, 45 μm, 50 μm, 55 μm, 60 μm, 65 μm, 70 μm, 75 μm, 80 μm, 85 μm, 90 μm, 10 μm, 100 μm, 105 μm, 110 μm, 120 μm, to about 125 μm, or any range derivable therein. The dry particle laser diffraction characterization methods were used to determine the particle size and distribution. A laser diffractometer with a disperser with the detection range from 0.1-875 μm was used to collect the particle size and distribution data. An optimal concentration of 0.1% was setup as the trigger condition and a feed rate of 50% and 3 bar.

In some aspects, the pharmaceutical composition may exhibit a Carr's Index is from about 5 to about 28, from about 5 to about 25, from about 5 to about 21, from about 5 to about 15, or from about 15 to about 25. The Carr's Index may be from about 2, 4, 5, 6, 7, 8, 9, 10, 11, 12, 13, 14, 15, 16, 17, 18, 19, 20, 21, 22, 23, 24, 25, 28, 30, 32, 35, 38, to about 40, or any range derivable therein. Carr's Index of the pharmaceutical composition may be determined by tapped density which is measured after a powder sample is subjected to mechanically tapping. The measurement procedure for bulk density and tapped density can be found in the US Pharmacopeia. Bulk density and tapped density can be used to calculate the Carr's compressibility index and Hausner ratio, which are measures of the propensity of a powder to flow and be compressed:

$$Compressibiliy index ( \% ) = \frac{tapped density - bulk density}{tapped density} \times 100$$

$${Hausner}^{′} s ratio = \frac{Tapped density}{bulk density}$$

The bulk density of the composition may be less than 5 g/cm³, less than 4 g/cm³, less than 3 g/cm³, less than 2.5 g/cm³, less than 2.25 g/cm³, less than 2 g/cm³, less than 1.75 g/cm³, or less than 1.5 g/cm³. The bulk density may be in a range from about 0.25 g/cm³, 0.5 g/cm³, 0.75 g/cm³, 1 g/cm³, 1.25 g/cm³, 1.5 g/cm³, 1.75 g/cm³, 2 g/cm³, 2.25 g/cm³, 2.5 g/cm³, 3 g/cm³, 3.5 g/cm³, 4 g/cm³, 4.5 g/cm³, to about 5 g/cm³, or any range derivable therein. The bulk density were measured using a graduate cylinder by gently pass a quantity of powder sufficient to complete the test through a U.S. standard sieve #18 or smaller. The agglomerates formed during storage were break up before test. Approximately 100 g±1.0% (RSD) of the test sample (m) weighed were passed to a dry graduated cylinder of 250 ml (readable to 2 ml) without compacting, and read the unsettled apparent volume (Vo) to the nearest graduated unit. Calculate the bulk density in (g/cm³) using the formula m/Vo. The tapped density is measured by mechanically tapping a graduated measuring cylinder containing the powder sample. Powder samples were proceeded to a 250 ml graduated cylinder (readable to 2 ml) and a settling apparatus capable of producing 250±15 taps/min, and bulk volume (Vo) was determined using abovementioned methods. 10, 500 and 1250 taps on the same powder sample were conducted and the corresponding volumes V10, V500 and V1250 were recorded. (If the difference between V500 and V1250 is less than or equal to 2 ml, V1250 is the tapped volume. If the difference between V500 and V1250 exceeds 2 ml, repeat in increments such as 1250 taps, until the difference between succeeding measurements is less than or equal to 2 ml.) Calculate the tapped density (g/cm³) using the formula m/V_{f} in which V_{f} is the final tapped volume.

Additionally, the present disclosure relates to the use of compositions of the API and co-former which may be used to prepare the pharmaceutical compositions. These compostions may be substantially in the liquid form. The amount of the composition that is in the liquid phase may be greater than about 50%, 60%, 70%, 80%, 90%, 95%, 96%, 97%, 98%, or 99%. In some embodiments, the composition comprises about 50%, 60%, 65%, 70%, 75%, 80%, 85%, 90%, 92.5%, 95%, 96%, 97%, 97.5%, 98%, 98.5%, 99%, 99.5%, to about 99.9%, or any range derivable therein of the composition substantially in the liquid phase. These composition are often characterized by their viscosity wherein the composition may have a viscosity from 0.1 mPa*s to 250 mPa*s, from about 0.25 mPa*s to 150 mPa*s, or from about 0.5 mPa*s to about 50 mPa*s. The viscosity of the liquid binder material may be from about 0.1 mPa*s, 0.25 mPa*s, 0.5 mPa*s, 1 mPa*s, 2.5 mPa*s, 5 mPa*s, 10 mPa*s, 25 mPa*s, 50 mPa*s, 75 mPa*s, 100 mPa*s, 125 mPa*s, 150 mPa*s, 175 mPa*s, 200 mPa*s, 225 mPa*s, to about 250 mPa*s, or any range derivable therein. The rheological measurements could be carried out using a rotational viscometer, where the molten samples are placed in the water or oil bath. The measurement range of the Viscometer from 10% to 100% full scale torque can be adjusted by selection of specific spindles and the rotational speed (0.3-100 RPM) for various molten physical mixtures.

In some aspects, the present pharmaceutical composition may be exhibit compressibility that makes the composition useful for the production of pharmaceutical dosage forms such as oral forms like capsules or tablets. The pharmaceutical composition may also be used in a powder-based additive manufacturing application such as vat photopolymerization, material jetting, binder jetting, powder-bed fusion, material extrusion, directed energy deposition, or sheet lamination like fused deposition modeling, binder spraying, or selective laser sintering. These 3D printing platforms may be used in pharmaceutical manufacturing and patient-specific personalized therapy to produce on-demand pharmaceutical compositions.

A. Active Pharmaceutical Ingredient

In some embodiments, the active pharmaceutical ingredient is classified using the Biopharmaceutical Classification System (BCS), originally developed by G. Amidon, which separates pharmaceuticals for oral administration into four classes depending on their aqueous solubility and their permeability through the cell lining of the gastrointestinal tract. According to the BCS, drug substances are classified as follows: Class I—High Permeability, High Solubility; Class II—High Permeability, Low Solubility; Class III—Low Permeability, High Solubility; and Class IV—Low Permeability, Low Solubility.

In particular, typical BCS Class II that may be incorporated into the present pharmaceutical compositions include but are not limited to anti-infectious drugs such as Albendazole, Acyclovir, Azithromycin, Cefdinir, Cefuroxime axetil, Chloroquine, Clarithromycin, Clofazimine, Diloxanide, Efavirenz, Fluconazole, Griseofulvin, Indinavir, Itraconazole, Ketoconazole, Lopinavir, Mebendazole, Nelfinavir, Nevirapine, Niclosamide, Praziquantel, Pyrantel, Pyrimethamine, Quinine, and Ritonavir. Antineoplastic drugs such as Bicalutamide, Cyproterone, Gefitinib, Imatinib, and Tamoxifen. Biologic and Immunologic Agents such as Cyclosporine, Mycophenolate mofetil, Tacrolimus. Cardiovascular Agents such as Acetazolamide, Atorvastatin, Benidipine, Candesartan cilexetil, Carvedilol, Cilostazol, Clopidogrel, Ethylicosapentate, Ezetimibe, Fenofibrate, Irbesartan, Manidipine, Nifedipine, Nilvadipine, Nisoldipine, Simvastatin, Spironolactone, Telmisartan, Ticlopidine, Valsartan, Verapamil, Warfarin. Central Nervous System Agents such as Acetaminophen, Amisulpride, Aripiprazole, Carbamazepine, Celecoxib, Chlorpromazine, Clozapine, Diazepam, Diclofenac, Flurbiprofen, Haloperidol, Ibuprofen, Ketoprofen, Lamotrigine, Levodopa, Lorazepam, Meloxicam, Metaxalone, Methylphenidate, Metoclopramide, Nicergoline, Naproxen, Olanzapine, Oxcarbazepine, Phenytoin, Quetiapine Risperidone, Rofecoxib, and Valproic acid. Dermatological Agents such as Isotretinoin—Endocrine and Metabolic Agents such as Dexamethasone, Danazol, Epalrestat, Gliclazide, Glimepiride, Glipizide, Glyburide (glibenclamide), levothyroxine sodium, Medroxyprogesterone, Pioglitazone, and Raloxifene. Gastrointestinal Agents such as Mosapride, Orlistat, Cisapride, Rebamipide, Sulfasalazine, Teprenone, and Ursodeoxycholic Acid. Respiratory Agents such as Ebastine, Hydroxyzine, Loratadine, and Pranlukast. However, the skilled person will be well aware of other BCS class II drugs which can be used with the pharmaceutical compositions described herein.

Additionally, BCS class III drugs that may be incorporated into the present pharmaceutical compositions include but are not limited to cimetidine, acyclovir, atenolol, ranitidine, abacavir, captopril, chloramphenicol, codeine, colchicine, dapsone, ergotamine, kanamycin, tobramycin, tigecycline, zanamivir, hydralazine, hydrochlorothiazide, levothyroxine, methyldopa, paracetamol, propylthiouracil, i pyridostigmine, cloxacillin, thiamine, benzimidazole, didanosine, ethambutol, ethosuximide, folic acid, nicotinamide, nifurtimox, and salbutamol sulfate. However, the skilled person will be well aware of other BCS class III drugs which can be used with the pharmaceutical compositions described herein.

Additionally, BCS class IV drugs that may be incorporated into the present pharmaceutical compositions include but are not limited to hydrochlorothiazide, furosemide, cyclosporin A, itraconazole, indinavir, nelfinavir, ritonavir, saquinavir, nitrofurantoin, albendazole, acetazolamide, azithromycin, senna, azathioprine, chlorthalidone, BI-639667, rifabutin, paclitaxel, curcumin, etoposide, neomycin, methotrexate, atazanavir sulfate, Aprepitant, amphotericin B, amiodarone, or mesalamine. However, the skilled person will be well aware of other BCS class IV drugs which can be used with the pharmaceutical compositions described herein.

While the pharmaceutical compositions and methods described herein can be applied to any BCS class of drugs, BCS class II and IV are of interest for the pharmaceutical compositions described herein. Additionally, other API that are of specific consideration are those that are high melting point drugs such as a drug that has a melting point of greater than 200° C. Alternatively, the API used herein may have a melting point from about 25° C. to about 1,000° C., from about 100° C. to about 750° C., or from about 200° C. to about 500° C. In particular, the melting point may be greater than 200° C., 250° C., 300° C., 400° C., 500° C., 300° C., 700° C., 750° C., 800° C., 900° C., or 1,000° C.

In some aspects, the present methods may be used to formulate one or more poorly soluble API such as deferasirox, etravirine, indomethacin, posaconazole, and ritonavir. Etravirine is a neutral active pharmaceutical ingredient and may be used as a model for other neutral API. Deferasirox and indomethacin is a weak acid API and may be used as a model for other weak acid APIs. Posaconazole, itraconazole, and ritonavir are weak base APIs and may be used as models for other weak base APIs.

Suitable API may be any poorly water-soluble, biologically API or a salt, isomer, ester, ether or other derivative thereof, which include, but are not limited to, anticancer agents, antiallergic agents, antifungal agents, psychiatric agents such as analgesics, consciousness level-altering agents such as anesthetic agents or hypnotics, nonsteroidal anti-inflammatory agents (NSAIDS), anthelminthics, antiacne agents, antianginal agents, antiarrhythmic agents, anti-asthma agents, antibacterial agents, anti-benign prostate hypertrophy agents, anticoagulants, antidepressants, antidiabetics, antiemetics, antiepileptics, antigout agents, antihypertensive agents, anti-inflammatory agents, antimalarials, antimigraine agents, antimuscarinic agents, antineoplastic agents, antiobesity agents, antiosteoporosis agents, antiparkinsonian agents, antiproliferative agents, antiprotozoal agents, antithyroid agents, antitussive agent, anti-urinary incontinence agents, antiviral agents, anxiolytic agents, appetite suppressants, beta-blockers, cardiac inotropic agents, chemotherapeutic drugs, cognition enhancers, contraceptives, corticosteroids, Cox-2 inhibitors, diuretics, erectile dysfunction improvement agents, expectorants, gastrointestinal agents, histamine receptor antagonists, immunosuppressants, keratolytics, lipid regulating agents, leukotriene inhibitors, macrolides, muscle relaxants, neuroleptics, nutritional agents, opioid analgesics, protease inhibitors, or sedatives.

Non-limiting examples of the API may include 7-Methoxypteridine, 7-Methylpteridine, abacavir, abafungin, abarelix, acebutolol, acenaphthene, acetaminophen, acetanilide, acetazolamide, acetohexamide, acetretin, acrivastine, adenine, adenosine, alatrofloxacin, albuterol, alclofenac, aldesleukin, alemtuzumab, alfuzosin, alitretinoin, allobarbital, allopurinol, all-transretinoic acid (ATRA), aloxiprin, alprazolam, alprenolol, altretamine, amifostine, amiloride, aminoglutethimide, aminopyrine, amiodarone, amitriptyline, amlodipine, amobarbital, amodiaquine, amoxapine, amphetamine, amphotericin, amphotericin B, ampicillin, amprenavir, amsacrine, amylnitrate, amylobarbitone, anastrozole, anrinone, anthracene, anthracyclines, aprobarbital, aripiprazole, arsenic trioxide, asparaginase, aspirin, astemizole, atenolol, atorvastatin, atovaquone, atrazine, atropine, atropine azathioprine, auranofin, azacitidine, azapropazone, azathioprine, azintamide, azithromycin, aztreonum, baclofen, barbitone, BCG live, beclamide, beclomethasone, bendroflumethiazide, benezepril, benidipine, benorylate, benperidol, bentazepam, benzamide, benzanthracene, benzathine penicillin, benzhexol, benznidazole, benzodiazepines, benzoic acid, bephenium hydroxynaphthoate, betamethasone, bevacizumab (avastin), bexarotene, bezafibrate, bicalutamide, bifonazole, biperiden, bisacodyl, bisantrene, bleomycin, bleomycin, bortezomib, brinzolamide, bromazepam, bromocriptine mesylate, bromperidol, brotizolam, budesonide, bumetanide, bupropion, busulfan, butalbital, butamben, butenafine, butobarbitone, butobarbitone (butethal), butoconazole, butoconazole nitrate, butylparaben, caffeine, calcifediol, calciprotriene, calcitriol, calusterone, cambendazole, camphor, camptothecin, camptothecin analogs, candesartan, capecitabine, capsaicin, captopril, carbamazepine, carbimazole, carbofuran, carboplatin, carbromal, carimazole, carmustine, cefamandole, cefazolin, cefixime, ceftazidime, cefuroxime axetil, celecoxib, cephradine, cerivastatin, cetrizine, cetuximab, chlorambucil, chloramphenicol, chlordiazepoxide, chlormethiazole, chloroquine, chlorothiazide, chlorpheniramine, chlorproguanil, chlorpromazine, chlorpropamide, chlorprothixene, chlorpyrifos, chlortetracycline, chlorthalidone, chlorzoxazone, cholecalciferol, chrysene, cilostazol, cimetidine, cinnarizine, cinoxacin, ciprofibrate, ciprofloxacin, cisapride, cisplatin, citalopram, cladribine, clarithromycin, clemastine fumarate, clioquinol, clobazam, clofarabine, clofazimine, clofibrate, clomiphene citrate, clomipramine, clonazepam, clopidogrel, clotiazepam, clotrimazole, clotrimazole, cloxacillin, clozapine, cocaine, codeine, colchicine, colistin, conjugated estrogens, corticosterone, cortisone, cortisone acetate, cyclizine, cyclobarbital, cyclobenzaprine, cyclobutane-spirobarbiturate, cycloethane-spirobarbiturate, cycloheptane-spirobarbiturate, cyclohexane-spirobarbiturate, cyclopentane-spirobarbiturate, cyclophosphamide, cyclopropane-spirobarbiturate, cycloserine, cyclosporin, cyproheptadine, cyproheptadine, cytarabine, cytosine, dacarbazine, dactinomycin, danazol, danthron, dantrolene sodium, dapsone, darbepoetin alfa, darodipine, daunorubicin, decoquinate, dehydroepiandrosterone, delavirdine, demeclocycline, denileukin, deoxycorticosterone, desoxymethasone, dexamethasone, dexamphetamine, dexchlorpheniramine, dexfenfluramine, dexrazoxane, dextropropoxyphene, diamorphine, diatrizoicacid, diazepam, diazoxide, dichlorophen, dichlorprop, diclofenac, dicumarol, didanosine, diflunisal, digitoxin, digoxin, dihydrocodeine, dihydroequilin, dihydroergotamine mesylate, diiodohydroxyquinoline, diltiazem, diloxamide furoate, dimenhydrinate, dimorpholamine, dinitolmide, diosgenin, diphenoxylate, diphenyl, dipyridamole, dirithromycin, disopyramide, disulfiram, diuron, docetaxel, domperidone, donepezil, doxazosin, doxazosin, doxorubicin (neutral), doxorubicin, doxycycline, dromostanolone propionate, droperidol, dyphylline, echinocandins, econazole, econazole nitrate, efavirenz, ellipticine, enalapril, enlimomab, enoximone, epinephrine, epipodophyllotoxin derivatives, epirubicin, epoetinalfa, eposartan, equilenin, equilin, ergocalciferol, ergotamine tartrate, erlotinib, erythromycin, estradiol, estramustine, estriol, estrone, ethacrynic acid, ethambutol, ethinamate, ethionamide, ethopropazine, ethyl-4-aminobenzoate (benzocaine), ethylparaben, ethinylestradiol, etodolac, etomidate, etoposide, etretinate, exemestane, felbamate, felodipine, fenbendazole, fenbuconazole, fenbufen, fenchlorphos, fenclofenac, fenfluramine, fenofibrate, fenoldepam, fenoprofen calcium, fenoxycarb, fenpiclonil, fentanyl, fenticonazole, fexofenadine, filgrastim, finasteride, flecamide acetate, floxuridine, fludarabine, fluconazole, fluconazole, flucytosine, fludioxonil, fludrocortisone, fludrocortisone acetate, flufenamic acid, flunanisone, flunarizine, flunisolide, flunitrazepam, fluocortolone, fluometuron, fluorene, fluorouracil, fluoxetine, fluoxymesterone, flupenthixol decanoate, fluphenthixol decanoate, flurazepam, flurbiprofen, fluticasone propionate, fluvastatin, folic acid, fosenopril, fosphenytoin sodium, frovatriptan, furosemide, fulvestrant, furazolidone, gabapentin, G-BHC (Lindane), gefitinib, gemcitabine, gemfibrozil, gemtuzumab, glafenine, glibenclamide, gliclazide, glimepiride, glipizide, glutethimide, glyburide, Glyceryltrinitrate (nitroglycerin), goserelin acetate, grepafloxacin, griseofulvin, guaifenesin, guanabenz acetate, guanine, halofantrine, haloperidol, hydrochlorothiazide, heptabarbital, heroin, hesperetin, hexachlorobenzene, hexethal, histrelin acetate, hydrocortisone, hydroflumethiazide, hydroxyurea, hyoscyamine, hypoxanthine, ibritumomab, ibuprofen, idarubicin, idobutal, ifosfamide, ihydroequilenin, imatinib mesylate, imipenem, indapamide, indinavir, indomethacin, indoprofen, interferon alfa-2a, interferon alfa-2b, iodamide, iopanoic acid, iprodione, irbesartan, irinotecan, isavuconazole, isocarboxazid, isoconazole, isoguanine, isoniazid, isopropylbarbiturate, isoproturon, isosorbide dinitrate, isosorbide mononitrate, isradipine, itraconazole, itraconazole, itraconazole (Itra), ivermectin, ketoconazole, ketoprofen, ketorolac, khellin, labetalol, lamivudine, lamotrigine, lanatoside C, lanosprazole, L-DOPA, leflunomide, lenalidomide, letrozole, leucovorin, leuprolide acetate, levamisole, levofloxacin, lidocaine, linuron, lisinopril, lomefloxacin, lomustine, loperamide, loratadine, lorazepam, lorefloxacin, lormetazepam, losartan mesylate, lovastatin, lysuride maleate, Maprotiline, mazindol, Meclizine, meclofenamic acid, medazepam, medigoxin, medroxyprogesterone acetate, mefenamic acid, Mefloquine, megestrol acetate, melphalan, mepenzolate bromide, meprobamate, meptazinol, mercaptopurine, mesalazine, mesna, mesoridazine, mestranol, methadone, methaqualone, methocarbamol, methoin, methotrexate, methoxsalen, methsuximide, methyclothiazide, methylphenidate, methylphenobarbitone, methyl-p-hydroxybenzoate, methylprednisolone, methyltestosterone, methyprylon, methysergide maleate, metoclopramide, metolazone, metoprolol, metronidazole, Mianserin, miconazole, midazolam, mifepristone, miglitol, minocycline, minoxidil, mitomycin C, mitotane, mitoxantrone, mofetilmycophenolate, molindone, montelukast, morphine, Moxifloxacin, nabumetone, nadolol, nalbuphine, nalidixic acid, nandrolone, naphthacene, naphthalene, naproxen, naratriptan, natamycin, nelarabine, nelfinavir, nevirapine, nicardipine, niclosamide, nicotin amide, nicotinic acid, nicoumalone, nifedipine, nilutamide, nimodipine, nimorazole, nisoldipine, nitrazepam, nitrofurantoin, nitrofurazone, nizatidine, nofetumomab, norethisterone, norfloxacin, norgestrel, nortriptyline, nystatin, oestradiol, ofloxacin, olanzapine, omeprazole, omoconazole, ondansetron, oprelvekin, ornidazole, oxaliplatin, oxamniquine, oxantelembonate, oxaprozin, oxatomide, oxazepam, oxcarbazepine, oxfendazole, oxiconazole, oxprenolol, oxyphenbutazone, oxyphencyclimine, paclitaxel, palifermin, pamidronate, p-aminosalicylic acid, pantoprazole, paramethadione, paroxetine, pegademase, pegaspargase, pegfilgrastim, pemetrexeddisodium, penicillamine, pentaerythritol tetranitrate, pentazocin, pentazocine, pentobarbital, pentobarbitone, pentostatin, pentoxifylline, perphenazine, perphenazine pimozide, perylene, phenacemide, phenacetin, phenanthrene, phenindione, phenobarbital, phenolbarbitone, phenolphthalein, phenoxybenzamine, phenoxybenzamine, phenoxymethyl penicillin, phensuximide, phenylbutazone, phenytoin, pindolol, pioglitazone, pipobroman, piroxicam, pizotifen maleate, platinum compounds, plicamycin, polyenes, polymyxin B, porfimersodium, posaconazole (Posa), pramipexole, prasterone, pravastatin, praziquantel, prazosin, prazosin, prednisolone, prednisone, primidone, probarbital, probenecid, probucol, procarbazine, prochlorperazine, progesterone, proguanil, promethazine, propofol, propoxur, propranolol, propylparaben, propylthiouracil, prostaglandin, pseudoephedrine, pteridine-2-methyl-thiol, pteridine-2-thiol, pteridine-4-methyl-thiol, pteridine-4-thiol, pteridine-7-methyl-thiol, pteridine-7-thiol, pyrantelembonate, pyrazinamide, pyrene, pyridostigmine, pyrimethamine, quetiapine, quinacrine, quinapril, quinidine, quinidine sulfate, quinine, quininesulfate, rabeprazole sodium, ranitidine rasburicase, ravuconazole, repaglinide, reposal, reserpine, retinoids, rifabutine, rifampicin, rifapentine, rimexolone, risperidone, ritonavir, rituximab, rizatriptan benzoate, rofecoxib, ropinirole, rosiglitazone, saccharin, salbutamol, salicylamide, salicylic acid, saquinavir, sargramostim, secbutabarbital, secobarbital, sertaconazole, sertindole, sertraline, simvastatin, sirolimus, sorafenib, sparfloxacin, spiramycin, spironolactone, stanolone, stanozolol, stavudine, stilbestrol, streptozocin, strychnine, sulconazole, sulconazole nitrate, sulfacetamide, sulfadiazine, sulfamerazine, sulfamethazine, sulfamethoxazole, sulfanilamide, sulfathiazole, sulindac, sulphabenzamide, sulphacetamide, sulphadiazine, sulphadoxine, sulphafurazole, sulphamerazine, sulpha-methoxazole, sulphapyridine, sulphasalazine, sulphinpyrazone, sulpiride, sulthiame, sumatriptan succinate, sunitinib maleate, tacrine, tacrolimus, talbutal, tamoxifen citrate, tamulosin, targretin, taxanes, tazarotene, telmisartan, temazepam, temozolomide, teniposide, tenoxicam, terazosin, terazosin, terbinafine, terbutaline sulfate, terconazole, terfenadine, testolactone, testosterone, tetracycline, tetrahydrocannabinol, tetroxoprim, thalidomide, thebaine, theobromine, theophylline, thiabendazole, thiamphenicol, thioguanine, thioridazine, thiotepa, thotoin, thymine, tiagabine, tibolone, ticlopidine, tinidazole, tioconazole, tirofiban, tizanidine, tolazamide, tolbutamide, tolcapone, topiramate, topotecan, toremifene, tositumomab, tramadol, tranilast, trastuzumab, trazodone, tretinoin, triamcinolone, triamterene, triazolam, triazoles, triflupromazine, trimethoprim, trimipramine maleate, triphenylene, troglitazone, tromethamine, tropicamide, trovafloxacin, tybamate, ubidecarenone (coenzyme Q10), undecenoic acid, uracil, uracil mustard, uric acid, valproic acid, valrubicin, valsartan, vancomycin, venlafaxine, vigabatrin, vinbarbital, vinblastine, vincristine, vinorelbine, voriconazole, xanthine, zafirlukast, zidovudine, zileuton, zoledronate, zoledronic acid, zolmitriptan, zolpidem, and zopiclone.

In particular aspects, the API may be busulfan, taxane, or other anticancer agents; alternatively, itraconazole (Itra) and posaconazole (Posa) or other members of the general class of azole compounds. Exemplary antifungal azoles include a) imidazoles such as miconazole, ketoconazole, clotrimazole, econazole, omoconazole, bifonazole, butoconazole, fenticonazole, isoconazole, oxiconazole, sertaconazole, sulconazole and tioconazole, b) triazoles such as fluconazole, itraconazole, isavuconazole, ravuconazole, Posaconazole, voriconazole, terconazole, and c) thiazoles such as abafungin. Other API that may be used with this approach include, but are not limited to, hyperthyroid drugs such as carbimazole, anticancer agents like cytotoxic agents such as epipodophyllotoxin derivatives, taxanes, bleomycin, anthracyclines, as well as platinum compounds and camptothecin analogs. The following API may also include other antifungal antibiotics, such as poorly water-soluble echinocandins, polyenes (e.g., Amphotericin B and Natamycin) as well as antibacterial agents (e.g., polymyxin B and colistin), and anti-viral drugs. The API may also include a psychiatric agent such as an antipsychotic, anti-depressive agent, or analgesic and/or tranquilizing agents such as benzodiazepines. The API may also include a consciousness level-altering agent or an anesthetic agent, such as propofol. The present compositions and the methods of making them may be used to prepare a pharmaceutical composition with the appropriate pharmacokinetic properties for use as therapeutics.

B. Co-Former

In some aspects, the present disclosure comprises one or more excipients formulated into pharmaceutical compositions as co-former with the active pharmaceutical ingredient to form a co-crystal. The co-former may be an excipient such as pharmaceutically acceptable carriers that are relatively inert substances used to facilitate administration or delivery of an API into a subject or used to facilitate the processing of an API into drug formulations that can be used pharmaceutically for delivery to the site of action in a subject. Non-limiting examples of excipients that may be used in the co-crystals include vitamins, polymer-carriers, stabilizing agents, surfactants, surface modifiers, solubility enhancers, buffers, encapsulating agents, antioxidants, preservatives, nonionic wetting or clarifying agents, viscosity-increasing agents, and absorption-enhancing agents.

In some embodiments, the compositions are substantially, essentially, or entirely free of any other excipient other than the co-former. In other embodiments, the composition comprises one or more excipients. In other embodiments, the co-crystals comprise a co-former which is a second active pharmaceutical ingredient rather than an excipient. In one aspect, the present disclosure relates to co-formers that contain one or more carboxylic acids. These co-former may be an aliphatic carbon group having from 1 to 18 carbon atoms with at least —CO₂H groups. In other aspects, the co-former may be a vitamin or a precursor molecule to a vitamin. These co-former may also include a vitamin derivative. Some non-limiting examples of vitamins, vitamin precursors, or vitamin derivatives may include nicotinamide, retinol, thiamine, riboflavin, pantothenic acid, pyridoxine, biotin, folic acid, cyanocobalamin, ascorbic acid, cholecalciferol, tocopherols, or phylloquinone.

The co-former may interact with the active pharmaceutical ingredient though one or more non-covalent interactions. These non-covalent interactions may include ionic interactions, hydrogen bonding, halogen bonding, van der Waals forces, π-π interactions, or hydrophobic effects. The interactions between the co-formers and active pharmaceutical ingredients may comprise two, three, four, five, or six non-covalent interactions which may be the same or a different type of non-covalent interaction. The co-former may interact with the active pharmaceutical ingredients in such a way that it modifies the properties of the active pharmaceutical ingredient including changing its solubility profile. The co-former itself may be sparingly soluble, sensitive to the environment such as the pH or the temperature.

C. Excipient

In some aspects, the present disclosure comprises one or more excipients formulated into pharmaceutical compositions such as a pharmaceutically acceptable thermoplastic polymer. An “excipient” refers to pharmaceutically acceptable carriers that are relatively inert substances used to facilitate administration or delivery of an API into a subject or used to facilitate the processing of an API into drug formulations that can be used pharmaceutically for delivery to the site of action in a subject. Non-limiting examples of excipients include polymer-carriers, stabilizing agents, surfactants, surface modifiers, solubility enhancers, buffers, encapsulating agents, antioxidants, preservatives, nonionic wetting or clarifying agents, viscosity-increasing agents, and absorption-enhancing agents. In some embodiments, the pharmaceutical composition is substantially, essentially, or entirely free of any other excipient.

1. Fillers

In some aspects, the pharmaceutical composition may further comprise one or more inorganic or organic material that may be used to bulk up a composition to obtain an effective amount of the compound. The filler may be an inert inorganic or organic compound such as a salt like a calcium, magnesium, sodium, or potassium salt or a sulfate, chloride, or nitrate salt. Commonly used organic compounds include carbohydrates, sugars, and sugar derivatives such as mannitol, lactose, starch, or cellulose.

Furthermore, the pharmaceutical compositions described herein have a concentration of filler ranging from about 1% to about 99% w/w. In some embodiments, the amount of each absorbent is from about 1% to about 99% w/w, from about 25% to about 98% w/w, 50% to about 98% w/w, or 75% to about 97% w/w. The amount of each absorbent may be from about 10%, 25%, 30%, 35%, 40%, 45%, 50%, 55%, 60%, 65%, 70%, 75%, 80%, 85%, 90%, 92.5%, 95%, 96%, 97%, 98%, to about 99%, or any range derivable therein. In some embodiments, the pharmaceutical composition is substantially, essentially, or entirely free of any other fillers.

2. Other Excipients

In some aspects, the present disclosure provides pharmaceutical compositions that may further comprise one or more additional excipients. The excipients (also called adjuvants) that may be used in the presently disclosed compositions and composites, while potentially having some activity in their own right, for example, antioxidants, are generally defined for this application as compounds that enhance the efficiency and/or efficacy of the active pharmaceutical ingredient. It is also possible to have more than one active pharmaceutical ingredient in a given solution so that the particles formed contain more than one active pharmaceutical ingredient.

Any pharmaceutically acceptable excipient known to those of skill in the art may be used to produce the pharmaceutical compositions disclosed herein. Examples of excipients for use with the present disclosure include, lactose, glucose, starch, calcium carbonate, kaolin, crystalline cellulose, silicic acid, water, simple syrup, glucose solution, starch solution, gelatin solution, carboxymethyl cellulose, shellac, methyl cellulose, polyvinyl pyrrolidone, dried starch, sodium alginate, powdered agar, calcium carmelose, a mixture of starch and lactose, sucrose, butter, hydrogenated oil, a mixture of a quaternary ammonium base and sodium lauryl sulfate, glycerine and starch, lactose, bentonite, colloidal silicic acid, talc, stearates, and polyethylene glycol, sorbitan esters, polyoxyethylene sorbitan fatty acid esters, polyoxyethylene alkyl ethers, poloxamers (polyethylene-polypropylene glycol block copolymers), sucrose esters, sodium lauryl sulfate, oleic acid, lauric acid, vitamin E TPGS, polyoxyethylated glycolysed glycerides, dipalmitoyl phosphadityl choline, glycolic acid and salts, deoxycholic acid and salts, sodium fusidate, cyclodextrins, polyethylene glycols, polyglycolyzed glycerides, polyvinyl alcohols, polyacrylates, polymethacrylates, polyvinylpyrrolidones, phosphatidyl choline derivatives, cellulose derivatives, biocompatible polymers selected from poly(lactides), poly(glycolides), poly(lactide-co-glycolides), poly(lactic acid)s, poly(glycolic acid)s, poly(lactic acid-co-glycolic acid)s and blends, combinations, and copolymers thereof.

As stated, excipients and adjuvants may be used in the pharmaceutical composition to enhance the efficacy and efficiency of the active pharmaceutical ingredient in the pharmaceutical composition. Additional non-limiting examples of compounds that can be included are binders, carriers, cryoprotectants, lyoprotectants, surfactants, fillers, stabilizers, polymers, protease inhibitors, antioxidants, bioavailability enhancers, and absorption enhancers. The excipients may be chosen to modify the intended function of the active ingredient by improving flow, or bioavailability, or to control or delay the release of the API. Specific nonlimiting examples include sucrose, trehalose, Span 80, Span 20, Tween 80, Brij 35, Brij 98, Pluronic, sucroester 7, sucroester 11, sucroester 15, sodium lauryl sulfate (SLS, sodium dodecyl sulfate. SDS), dioctyl sodium sulphosuccinate (DSS, DOSS, dioctyl docusate sodium), oleic acid, laureth-9, laureth-8, lauric acid, vitamin E TPGS, Cremophor® EL, Cremophor® RH, Gelucire® 50/13, Gelucire® 53/10, Gelucire® 44/14, Labrafil®, Solutol® HS, dipalmitoyl phosphatidyl choline, glycolic acid and salts, deoxycholic acid and salts, sodium fusidate, cyclodextrins, polyethylene glycols, Labrasol®, polyvinyl alcohols, polyvinyl pyrrolidones, and tyloxapol.

The stabilizing carrier may also contain various functional excipients, such as: hydrophilic polymer, antioxidant, super-disintegrant, surfactant including amphiphilic molecules, wetting agent, stabilizing agent, retardant, similar functional excipient, or a combination thereof, and plasticizers including citrate esters, polyethylene glycols, PG, triacetin, diethyl phthalate, castor oil, and others known to those of ordinary skill in the art. Extruded material may also include an acidifying agent, adsorbent, alkalizing agent, buffering agent, colorant, flavorant, sweetening agent, diluent, opaquing agent, complexing agent, fragrance, preservative or a combination thereof.

Compositions with enhanced solubility may comprise a mixture of the active pharmaceutical ingredient and an additive that enhances the solubility of the active pharmaceutical ingredient. Examples of such additives include but are not limited to surfactants, polymer-carriers, pharmaceutical carriers, thermal binders, or other excipients. A particular example may be a mixture of the active pharmaceutical ingredient with a surfactant or surfactant, the active pharmaceutical ingredient with a polymer or polymers, or the active pharmaceutical ingredient with a combination of a surfactant and polymer carrier or surfactants and polymer-carriers. A further example is a composition where the active pharmaceutical ingredient is a derivative or analog thereof.

In some embodiments, the pharmaceutical compositions may further comprise one or more surfactants. Surfactants that can be used in the disclosed pharmaceutical compositions to enhance solubility include those known to a person of ordinary skill. Some particular non-limiting examples of such surfactants include but are not limited to sodium dodecyl sulfate, dioctyl docusate sodium, Tween 80, Span 20, Cremophor® EL or Vitamin E TPGS.

Solubility can be indicated by peak solubility, which is the highest concentration reached of a species of interest over time during a solubility experiment conducted in a specified medium at a given temperature. The enhanced solubility can be represented as the ratio of peak solubility of the agent in a pharmaceutical composition of the present disclosure compared to peak solubility of the reference standard agent under the same conditions. Preferably, an aqueous buffer with a pH in the range of from about pH 4 to pH 8, about pH 5 to pH 8, about pH 6 to pH 7, about pH 6 to pH 8, or about pH 7 to pH 8, such as, for example, pH 4.0, 4.5, 5.0, 5.5, 6.0, 6.2, 6.4, 6.6, 6.7, 6.8, 6.9, 7.0, 7.1, 7.2, 7.4, 7.6, 7.8, or 8.0, may be used for determining peak solubility. This peak solubility ratio can be about 2:1, 3:1, 4:1, 5:1, 6:1, 7:1, 8:1, 9:1, 10:1, 12:1, 15:1, 20:1, 25:1, 30:1, 35:1, 40:1, 45:1, 50:1, 55:1 or higher.

Compositions of the active pharmaceutical ingredient that enhance bioavailability may comprise a mixture of the active pharmaceutical ingredient and one or more pharmaceutically acceptable adjuvants that enhance the bioavailability of the active pharmaceutical ingredient. Examples of such adjuvants include but are not limited to enzyme inhibitors. Particular examples are such enzyme inhibitors include but are not limited to inhibitors that inhibit cytochrome P-450 enzyme and inhibitors that inhibit monoamine oxidase enzyme. Bioavailability can be indicated by the Cₘₐₓ or the AUC of the active pharmaceutical ingredient as determined during in vivo testing, where Cₘₐₓ is the highest reached blood level concentration of the active pharmaceutical ingredient over time of monitoring and AUC is the area under the plasma-time curve. Enhanced bioavailability can be represented as the ratio of Cₘₐₓ or the AUC of the active pharmaceutical ingredient in a pharmaceutical composition of the present disclosure compared to Cₘₐₓ or the AUC of the reference standard the active pharmaceutical ingredient under the same conditions. This Cₘₐₓ or AUC ratio reflecting enhanced bioavailability can be about 5:1, 6:1, 7:1, 8:1, 9:1, 10:1, 12:1, 15:1, 20:1, 25:1, 30:1, 35:1, 40:1, 45:1, 50:1, 55:1, 60:1, 65:1, 70:1, 75:1, 80:1, 85:1, 90:1, 95:1, 98:1, 99:1, 100:1 or higher.

In some aspects, the amount of the excipient in the pharmaceutical composition is from about 0.5% to about 20% w/w, from about 1% to about 10% w/w, from about 2% to about 8% w/w, or from about 3% to about 7% w/w. The amount of the excipient in the pharmaceutical composition comprises from about 0.5%, 1%, 1.5%, 2%, 2.5%, 3%, 3.5%, 4%, 4.5%, 5%, 5.5%, 6%, 6.5%, 7%, 7.5%, 8%, 9%, to about 10% w/w, or any range derivable therein, of the total pharmaceutical composition. In one embodiment, the amount of the excipient in the pharmaceutical composition is at 4% to 6% w/w of the total weight of the pharmaceutical composition.

### II. Dielectric Heating

Dielectric heating is a process where the dielectric material is heated under a certain radio frequency (RF), radio wave or microwave electromagnetic radiation. Such technologies have been applied for food processing and organic synthesis for many years (Kappe, 2008; Wiesbrock et al., 2004). In general, the molecular dipole rotation within the dielectric materials will generate heating at higher radio frequencies because of the polar molecules resonance frequency is close to microwave frequency (Kappe, 2004). Compared with the abovementioned approaches for poorly water soluble API improvement, dielectric heating offers several advantages including free of using organic solvents, rapid volumetric heating, energy-saving, and cost friendly (Zhou et al., 2003; Passerini et al., 2002). There are reports that pharmaceutical products were achieved using such techniques, but most of them are either dealing with the raw materials in raw powder forms (Kerč et al., 1998; Wen et al., 2004; Bergese et al., 2003; Moneghini et al., 2008) or solvents (Ahuja et al., 2020; Pagire et al., 2013) and are suitable for batch scale operation making the process lengthy. In this work, a novel dielectric heating method coupled with continuous mixing process such as an HME process was used to prepare the cocrystals loaded products with improved solubility, where the raw materials or drug products were subjected to the dielectric heating process. The crystalline structure was evaluated and characterized by several solid-state analytic methods.

In some embodiments, the dielectric heating process comprises exposing the pharmaceutical composition to electromagnetic radiation. The electrhaveomagnetic radiation may a specific frequency electromagnetic radiation. This specific frequency electromagnetic radiation may be a radio wave. The radio wave has a frequency from about 10 MHz to about 20 MHz. The radio wave may have a frequency from about 10 MHz, 11 MHz, 12 MHz, 13 MHz, 14 MHz, 15 MHz, 16 MHz, 17 MHz, 18 MHz, 19 MHz, to about 20 MHz, or any range derivable therein. In other aspect, the specific frequency electromagnetic radiation is a microwave. The microwave has a frequency greater than 100 MHz. In some embodiments, the frequency is from about 500 MHz to about 1,000 GHz, from about 1000 MHz to about 100 GHz, from about 1000 MHz to about 25 GHz, from about 1000 MHz to about 10 GHz, or from about 1000 MHz to about 3000 MHz. The frequency may be from about 1000 MHz, 2000 MHz, 3000 MHz, 4000 MHz, 5000 MHz, 7500 MHz, 10 GHz, 20 GHz, 30 GHz, 40 GHz, 50 GHz, 60 GHz, 70 GHz, 80 GHz, 90 GHz, 100 GHz, 250 GHz, 500 GHz, 750 GHz, to about 1000 GHz, or any range derivable therein.

The methods comprise using dielectric heating that provides a heating power to the mixture. The heating power may be from about 200 W to about 10 kW, from about 500 W to about 5 kW, from about 750 W to about 2 kW, or from about 800 W to about 1,500 W. The heating power may be from about 200 W, 300 W, 400 W, 500 W, 600 W, 700 W, 800 W, 900 W, 1000 W, 1100 W, 1200 W, 1300 W, 1400 W, 1500 W, 1600 W, 1750 W, 1800 W, 2000 W, 2500 W, 5 kW, 7.5 kW, to 10 kW, or any range derivable therein. Furthermore, the energy used in the dielectric heating may have a specific wavelength. The specific wavelength is greater than 1 mm. In some embodiments, the specific wavelength is from about 1 mm to about 1 m, from about 3 mm to about 300 mm, from about 50 mm to about 200 mm, or from about 100 mm to about 150 mm. The wavelength may be from about 1 mm, 5 mm, 10 mm, 25 mm, 50 mm, 75 mm, 100 mm, 200 mm, 300 mm, 400 mm, 500 mm, 750 mm, 1 m, 2.5 m, 5 m, to about 10 m, or any range therein.

The ability of a API and co-former combination to convert electromagnetic energy to heat under given frequency, wavenumber, and power is dominated by the loss factor, tan δ. The loss factor can be expressed as tan δ=dielectric loss/dielectric constant, where the dielectric loss represents the efficiency of converting electromagnetic radiation into heat, while the dielectric constant, also known as relative permittivity, refers to ability or power of the molecule to be polarized by the electromagnetic filed. The loss factor, tan δ may be from about 1*10⁻⁵ to about 0.1, from about 0.1 to about 0.5, from about 0.5 to about 1, or from about 1 to about 50. The tan δ may be from about 1*10⁻⁵, 0.1, 0.2, 0.5, 0.7, 1, 3, 5, to 25, or any range derivable therein. Furthermore, the API or co-former used in the dielectric heating may have a specific dielectric constant, which is greater than 1. In some embodiments, the specific dielectric constant is from about 1 to about 10, from about 8 to about 200, from about 100 to about 6000, or from about 1200 to about 100000. The dielectric constant may from about 100000, 10000, 6000, 1200, 500, 100, 80, 10, 2 to about 1, or any range therein.

### III. Mixing Techniques

In some embodiments, the methods comprises using a technique to achieve mixing of the composition or mixture subjected to dielectric heating. The mechanism of mixing is not important so long as that mixing leads to uniform distribution of the co-former and the API in the composition which has been exposed to dielectric heating. These methods may include mixing through extrusion, fluidized bed granulation, high shear granulation, propeller mixing, turbine mixing, high shear mixing, high pressure or ultrasonic homogenization. In one embodiment, the method may be performed through extrusion. Such process may include hot melt extrusion, hot melt granulation, melt mixing, spray congealing, sintering/curing, injection molding, or a thermokinetic mixing process such as the KinetiSol method. Similar thermal processing methods are described in LaFountaine et al., 2016a, Keen et al., 2013, Vynckier et al., 2014, Lang et al., 2014, Repka et al., 2007, Crowley et al., 2007, DiNunzio et al., 2010a, DiNunzio et al., 2010b, DiNunzio et al., 2010c, DiNunzio et al., 2010d, Hughey et al., 2010, Hughey et al., 2011, LaFountaine et al., 2016b, and Prasad et al., 2016, all of which are incorporated herein by reference. In some embodiments of these present disclosure, the pharmaceutical compositions may be prepared using a thermal process such as hot melt extrusion or hot melt granulation. In other embodiments, a fusion based process including thermokinetic mixing process such as those described at least in U.S. Pat. Nos. 8,486,423 and 9,339,440, the entire contents of which are herein incorporated by reference.

A non-limiting list of instruments which may be used to thermally process the pharmaceutical compositions described herein include hot melt extruders available from ThermoFisher, such as a minilab compounder, or Leistritz, such as a twin-screw extruder. Alternatively, a fusion-based high energy process instrument that does not require external heat input, including such as a thermokinetic mixer as described in U.S. Pat. Nos. 8,486,423 and 9,339,440 may be used to process the pharmaceutical composition.

In some aspects, the extruder may comprise modifying the temperature of the composition to one or more temperatures. These temperatures may be from about 0° C. to about 300° C. In some embodiments, the temperature is from about 10° C. to about 250° C. The temperature that may be used is from about 0° C., 5° C., 10° C., 15° C., 20° C., 22° C., 24° C., 25° C., 26° C., 28° C., 30° C., 35° C., 40° C., 45° C., 50° C., 55° C., 60° C., 65° C., 70° C., 75° C., 80° C., 90° C., 100° C., 110° C., 120° C., 125° C., 130° C., 135° C., 140° C., 145° C., 150° C., 160° C., 170° C., 180° C., 190° C., 200° C., 250° C., to about 300° C. or any range derivable therein.

In some aspects, the present disclosure provides using a processing technique such as an extruder. The extruder can comprise using a process which has a screw speed from about 10 rpm to about 400 rpm, from about 20 rpm to about 300 rpm, or from about 25 rpm to about 200 rpm. The screw speed may be from about 10 rpm, 20 rpm, 30 rpm, 40 rpm, 50 rpm, 60 rpm, 70 rpm, 80 rpm, 90 rpm, 100 rpm, 125 rpm, 150 rpm, 175 rpm, 200 rpm, 225 rpm, 250 rpm, 275 rpm, 300 rpm, 325 rpm, 350 rpm, 375 rpm, to about 400 rpm, or any range derivable therein. Similarly, the extruder may be used to prepare a yield of the extrudate that is from about 100 g/hr to about 2.5 kg/hr, from about 250 g/hr to about 2.0 kg/hr, or from about 300 g/hr to about 1.5 kg/hr. The yield of the extrudate may be from about 100 g/hr, 150 g/hr, 200 g/hr, 250 g/hr, 300 g/hr, 350 g/hr, 400 g/hr, 450 g/hr, 500 g/hr, 600 g/hr, 700 g/hr, 800 g/hr, 900 g/hr, 1 kg/hr, 1.2 kg/hr, 1.3 kg/hr, 1.4 kg/hr, 1.5 kg/hr, 1.6 kg/hr, 1.7 kg/hr, 1.8 kg/hr, 1.9 kg/hr, 2.0 kg/hr, 2.1 kg/hr, 2.2 kg/hr, 2.3 kg/hr, 2.4 kg/hr, to about 2.5 kg/hr, or any range derivable therein.

The extrudate produced following the extrusion process will generally comprise the API and the co-former. The extrudate may be in the form of granules of a desired mesh size or diameter, rods that can be cut and shaped into tablets, and films of a suitable thickness that shaped forms can be punched into suitable size and shape for administration. This extrudate may be used in further processing steps to yield the final pharmaceutical product or composition. The extrudate of the pharmaceutical composition may be dried, formed, milled, sieved, or any combination of these processes to obtain a final composition which may be administered to a patient. Such processes are routine and known in the art and include formulating the specific product to obtain a final pharmaceutical or nutraceutical product. Additionally, the extrudate of the pharmaceutical composition obtained may be processed using a tablet press to obtain a final table. Additionally, it may be milled and combined with one or more additional excipients to form a capsule or pressed into a table. The resultant pharmaceutical composition may also be dissolved in a solvent to obtain a syrup, a suspension, an emulsion, or a solution.

### IV. Definitions

The use of the word “a” or “an”, when used in conjunction with the term “comprising” in the claims and/or the specification, may mean “one,” but it is also consistent with the meaning of “one or more,” “at least one,” and “one or more than one.” As used herein “another” may mean at least a second or more.

As used herein, the terms “active pharmaceutical ingredient”, “drug”, “pharmaceutical”, “active agent”, “therapeutic agent”, and “therapeutically active agent” are used interchangeably to represent a compound which invokes a therapeutic or pharmacological effect in a human or animal and is used to treat a disease, disorder, or other condition. In some embodiments, these compounds have undergone and received regulatory approval for administration to a living creature.

The use of the term “or” in the claims is used to mean “and/or” unless explicitly indicated to refer to alternatives only or the alternatives are mutually exclusive. As used herein “another” may mean at least a second or more.

The terms “compositions,” “pharmaceutical compositions,” “formulations,” “pharmaceutical formulations,” “preparations”, and “pharmaceutical preparations” are used synonymously and interchangeably herein.

“Treating” or treatment of a disease or condition refers to executing a protocol, which may include administering one or more drugs to a patient, to alleviate signs or symptoms of the disease. Desirable effects of treatment include decreasing the rate of disease progression, ameliorating or palliating the disease state, and remission or improved prognosis. Alleviation can occur before signs or symptoms of the disease or condition appearing, as well as after their appearance. Thus, “treating” or “treatment” may include “preventing” or “prevention” of disease or undesirable condition. In addition, “treating” or “treatment” does not require complete alleviation of signs or symptoms, does not require a cure, and specifically includes protocols that have only a marginal effect on the patient.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, a reduction in the growth rate of the cancer, or prevention of metastasis. Treatment of cancer may also refer to prolonging the survival of a subject with cancer.

“Subject” and “patient” refer to either a human or non-human, such as primates, mammals, and vertebrates. In particular embodiments, the subject is a human.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds disclosed herein which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide, and calcium hydroxide. Acceptable organic bases include ethanolamine, diethanolamine, triethanolamine, tromethamine, N-methylglucamine, and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

The term “derivative thereof” refers to any chemically modified compound, wherein at least one of the compounds is modified by substitution of atoms or molecular groups or bonds. In one embodiment, a derivative thereof is a salt thereof. Salts are, for example, salts with suitable mineral acids, such as hydrohalic acids, sulfuric acid or phosphoric acid, for example, hydrochlorides, hydrobromides, sulfates, hydrogen sulfates or phosphates, salts with suitable carboxylic acids, such as optionally hydroxylated lower alkanoic acids, for example, acetic acid, glycolic acid, propionic acid, lactic acid or pivalic acid, optionally hydroxylated and/or oxo-substituted lower alkane dicarboxylic acids, for example, oxalic acid, succinic acid, fumaric acid, maleic acid, tartaric acid, citric acid, pyruvic acid, malic acid, ascorbic acid, and also with aromatic, heteroaromatic or araliphatic carboxylic acids, such as benzoic acid, nicotinic acid or mandelic acid, and salts with suitable aliphatic or aromatic sulfonic acids or N-substituted sulfamic acids, for example, methanesulfonates, benzenesulfonates, p-toluenesulfonates or N-cyclohexylsulfamates (cyclamates).

The term “amorphous” refers to a noncrystalline solid wherein the molecules are not organized in a definite lattice pattern. Alternatively, the term “crystalline” refers to a solid wherein the molecules in the solid have a definite lattice pattern. The crystallinity of the active pharmaceutical ingredient in the composition is measured by powder x-ray diffraction.

A “poorly soluble drug” refers to a drug that meets the requirements of the USP and BP solubility criteria of at least a sparingly soluble drug. The poorly soluble drug may be sparingly soluble, slightly soluble, very slightly soluble or practically insoluble. In a preferred embodiment, the drug is at least slightly soluble. In a more preferred embodiment, the drug is at least very slightly soluble. As defined by the USP and BP, a soluble drug is a drug which is dissolved from 10 to 30 part of solvent required per part of the solute, a sparingly soluble drug is a drug which is dissolved from 30 to 100 part of solvent required per part of the solute, a slightly soluble drug is a drug which is dissolved from 100 to 1,000 part of solvent required per part of the solute, a very slightly soluble drug is a drug which is dissolved from 1,000 to 10,000 part of solvent required per part of the solute, and a practically insoluble drug is a drug which is dissolved from 10,000 part of solvent required per part of solute. The solvent may be water that is at a pH from 1-7.5, preferably physiological pH.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

As used in this specification, the term “significant” (and any form of significance such as “significantly”) is not meant to imply statistical differences between two values but only to imply importance or the scope of the difference of the parameter.

Throughout this application, the term “about” is used to indicate that a value includes the inherent variation of error for the device, the method being employed to determine the value or the variation that exists among the study subjects or experimental studies. Unless another definition is applicable, the term “about” refers to ±10% of the indicated value.

As used herein, the term “substantially free of” or “substantially free” in terms of a specified component, is used herein to mean that none of the specified components has been purposefully formulated into a composition and/or is present only as a contaminant or in trace amounts. The total amount of all containments, by-products, and other material is present in that composition in an amount of less than 2%. The term “essentially free of” or “essentially free” is used to represent that the composition contains less than 1% of the specific component. The term “entirely free of” or “entirely free” contains less than 0.1% of the specific component.

The term “homogenous” is used to mean a composition in which the components are mixed in such a way that the components are uniformly distributed amongst the composition. In a preferred embodiment, the composition is uniformly distributed in such a manner that there are no regions of a single component that are greater than 1 μm or more preferably less than 0.1 μm. In one embodiment, the composition is so homogeneously mixed in such a manner that there are no atoms of the thermally conductive excipient are adjacent to another atom of the thermally conductive excipient.

The terms “substantially” or “approximately” as used herein may be applied to modify any quantitative comparison, value, measurement, or other representation that could permissibly vary without resulting in a change in the basic function to which it is related. Such an amount, when referring to the amount of the composition that is in the liquid phase, refers to an amount of composition such that the composition appears liquid or soft to the naked eye.

A temperature, when used without any other modifier, refers to room temperature, preferably 23° C. unless otherwise noted. An elevated temperature is a temperature that is more than 5° C. greater than room temperature; preferably more than 10° C. greater than room temperature.

The term “unit dose” refers to a formulation of the pharmaceutical composition such that the formulation is prepared in a manner sufficient to provide a single therapeutically effective dose of the active pharmaceutical ingredient to a patient in a single administration. Such unit dose formulations that may be used include but are not limited to a single tablet, capsule, or other oral formulations, or a single vial with a syringeable liquid or other injectable formulations. The resulting product can then undergo further downstream processing to create an intermediate product, such as granules, that can then be further formulated into a unit dose such as one prepared for oral delivery as tablets, capsules, three-dimensionally printed selective laser sintered (3DPSLS) or suspensions; pulmonary and nasal delivery; topical delivery as emulsions, ointments or creams; transdermal delivery; and parenteral delivery as suspensions, microemulsions or depot. In some forms, the final pharmaceutical composition that is produced is no longer a powder and is further produced as a homogenous final product. This final product has the capability of being processed into granules and being compressed or 3DPSLS into a final pharmaceutical unit dose form.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements and parameters.

Other objects, features, and advantages of the present disclosure will become apparent from the following detailed description. It should be understood, however, that the detailed description and the specific examples, while indicating preferred embodiments of the disclosure, are given by way of illustration only, since various changes and modifications within the spirit and scope of the disclosure will become apparent to those skilled in the art from this detailed description.

### IV. Examples

To facilitate a better understanding of the present disclosure, the following examples of specific embodiments are given. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure. In no way should the following examples be read to limit or define the entire scope of the disclosure.

### Example 1—Preparation of Formulations with HME and Dielectric Heating in Parallel

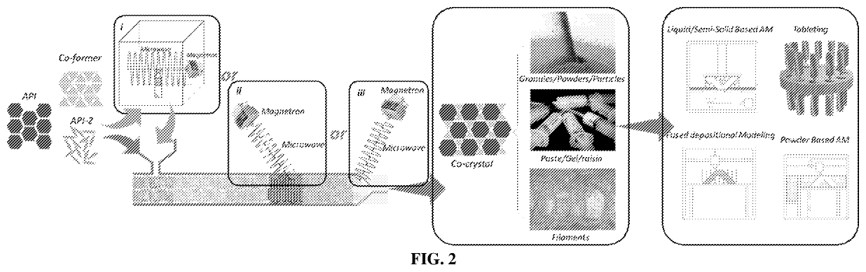

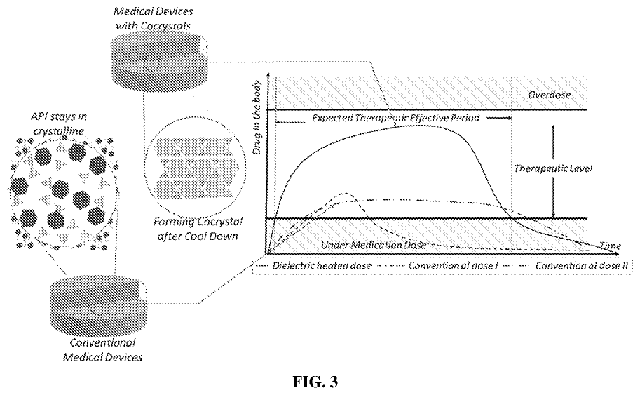

In one embodiment, the present disclosure provides that the APIs and co-formers can be processed via conjugating the dielectric heating and HME techniques (FIG. 2), where the dielectric heating will assist the melting or softening of the materials outside the barrel, then the soften or molten materials are fed into the barrel while the extrusion process will continuously convey and mix the materials to form the expected intermediate extrudates in solid free-flowing powder form. Besides, the extrusion process will be conducted at a decreased temperature from feeding zone to die to assist the phase transformation during extrusion. Then the obtained intermediate materials containing cocrystals will be subjected to further downstream process such as tableting, AM, or capsule filling either in the same production line by integrating all ancillaries or separately.

I. Formulation

The ibuprofen (IBU) was selected as API and nicotinamide (NTM) was selected as conformer. The API with co-former was physically mixed using a pestle and mortar.

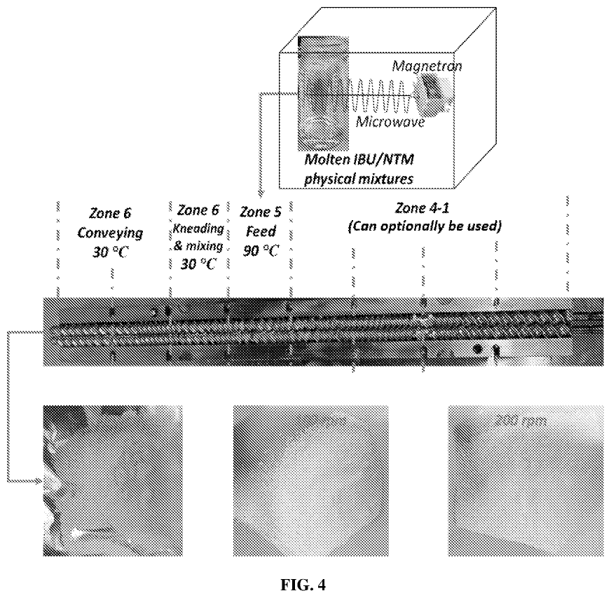

As shown in FIG. 2, 20 g of physically mixed powders was stored in a 20 ml vial then put in the middle of a Panasonic NE-1054F microwave oven, which generates the high-frequency microwave (2450 MHz). The heating power was 1000 W, the frequency was 2450 MHz and the wavelength was around 12.24 cm. The sample were heated until melt then take out from the oven and feed into the extruder. A L/D ratio=15 screw configuration (not including the feeding zone) with kneading blocks (L/D ratio=2) were used and the extruder were set at 30° C. and 50, 100, and 200 rpm. Physical mixture of IBU and NTM were dielectric heated and completely melt, then feeded into the extruder manually from zone 5 at around 5, 10, and 20 g/min, where zone 5 were set at 90° C. to prevent the solidification in feed zone (FIG. 4). In the current investigation, the zone 1-4 were not used, but those zones can optionally be used for future investigations such as for adding extra excipients or ingredients prior to formulating final dosage forms such as tablets.

Samples were collected and weighted. As shown in the FIG. 4, the higher extrusion speed, the larger particles size and broader particle size distribution will be. In addition, the yields of 50, 100, and 200 rpm was around 390, 534, and 1140 g/hr, which is dramatically higher than the conventional HME-crystallization process i.e. 37.8 g/h (Daurio et al., 2014) and 200 g/h (Kelly et al., 2012). These compositions and particle sizes may be further refinement of the HME process.

Powders were obtained from the dielectric heating-extrusion process, and some cocrystals obtained from cooling the dielectric heated (without extrusion) materials were also collected as reference.

II. Characterization of the Cocrystals

The produced cocrystals were collected and characterized using various solid states characterization techniques including DSC, PLM, FT-IR, and Raman spectroscopy.

i. PLM

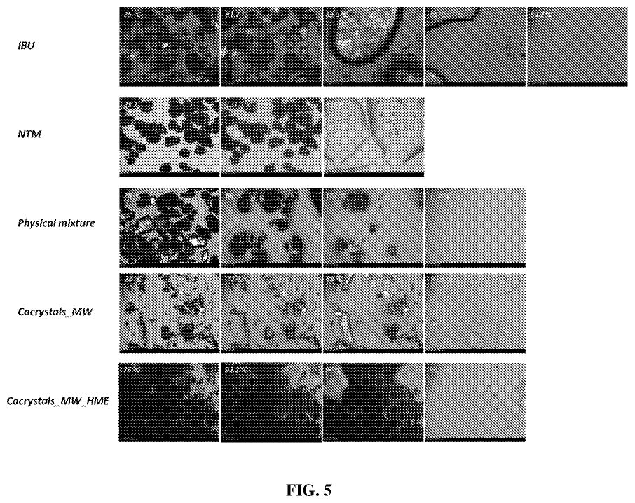

As shown in FIG. 5, the physical mixtures of each formulation were shown two different forms of crystals under the polarized light, on the contrary, the cocrystals showed single formed cocrystals. Further characterizations were conducted formation of cocrystals. Under the hot-stage PLM, the IBU melts around 82° C. while the NTM melts around 132° C. The physical mixture of IBU/NTM starts melt around 80° C., then the NTM dissolve into the molten matrix before 130° C. Both cocrystal samples starts melting around 90° C. and will be completely molten around 95° C.

ii. DSC

As shown in FIG. 6, the IBU and NTM showed eluting peak around 82.9° C. and 132.7° C., respectively; the physical mixture shows a melting peak of IBU around 77.8° C., but NTM melting peak around 92.1° C. which might due to the NTM dissolve into the molten IBU. The HME cocrystal only showed a single melting peak of 91.33° C., while the MW only cocrystal showed a small melting peak of 93.7° C., which matched the literature work (FIG. 6) IBU-NTM cocrystals manufactured via solvent methods.

iii. XRD

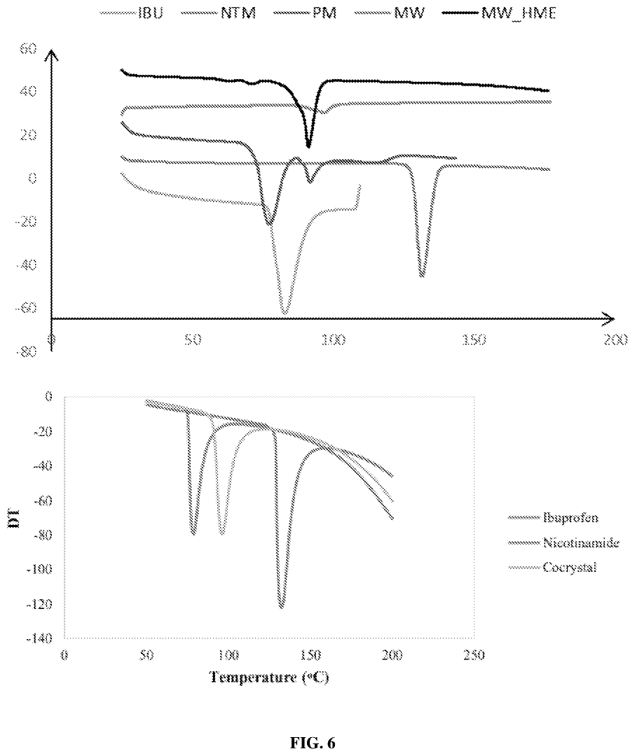

As shown in FIG. 7, the cocrystals new peaks at 2 theta range of 7.84, 13.2, 15.56, and 19.16, which was not seeing from neither NDP nor MLA crystals, which indicates the formation of the cocrystals. The ibuprofen shows peaks 2 theta range around 6.00, 12.1, 16.53, 17.48, 20.16, and 22.33; and nicotinamide appear at 2 theta range around 14.74, 22.20, 23.16, 25.26, 25.65, and 27.18. Both cocrystal samples show similar patters and the diffraction peaks around 2 theta angles=9.51, 12.64, 15.19, 15.57, 17.67, 18.76, 20.80, 21.82, 25.07, and 34.83.

iv. FTIR

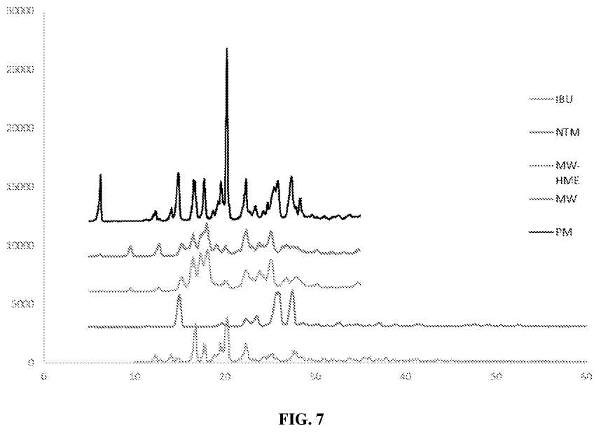

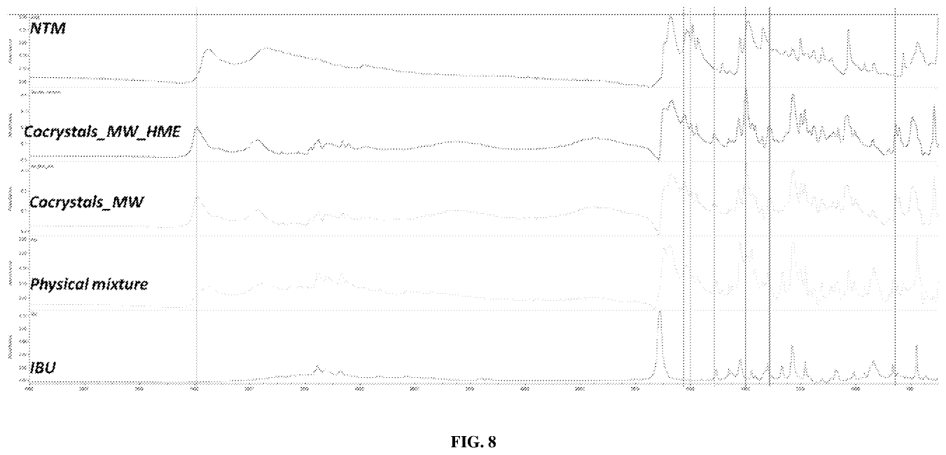

As shown in FIG. 8, both cocrystals samples showed similar spectrum, while the peaks were different with the IBU, NTM or the physical mixtures which confirms the formation of the cocrystals.

v. Raman

As shown in FIG. 9, both cocrystals samples showed similar spectrum, while the peaks were different with the IBU, NTM or the physical mixtures which confirms the formation of the cocrystals.

### Example 2—Preparation of Formulation when HME and Dielectric Heating are Coupled

In another embodiment the cocrystals can be produced via conjugate the dielectric heating and HME techniques, where the dielectric heat will assist the melting or soften the materials inside the barrel while the extrusion process will continuously convey and mix the materials to form the expected intermediate extrudates. Besides, the extrusion will be conducted at a decreasing temperature from feeding zone to die which will assist the phase transformation after dielectric heating zone. Then the obtained intermediate materials containing cocrystals will be subjected to further downstream process such as tableting, AM, or capsule filling.

I. Formulation

The acetylsalicylic acid (ASA), indomethacin (IND), ibuprofen (IBU), carbamazepine (CBZ), and nifedipine (NDP) were selected as APIs and nicotinamide (NTM) and malic acid (MLA) were selected as conformer. The formulation and ratio are listed in Table 1. The API with co-former was physically mixed using a pestle and mortar.

TABLE 1
Formulation composition of cocrystals

| | API (melting | Coformer (melting | |
|---|---|---|---|
| Formulation | point, ° C.) | point, ° C.) | Ratio |
| 1 | ASA (135° C.) | NTM (130° C.) | 1:1 |
| 2 | IND (158° C.) | NTM (130° C.) | 1:1 |
| 3 | IBU (80° C.) | NTM (130° C.) | 1:1 |
| 4 | CBZ (190° C.) | MLA (130° C.) | 1:1 |
| 5 | NDP (173° C.) | MLA (130° C.) | 1:1 |

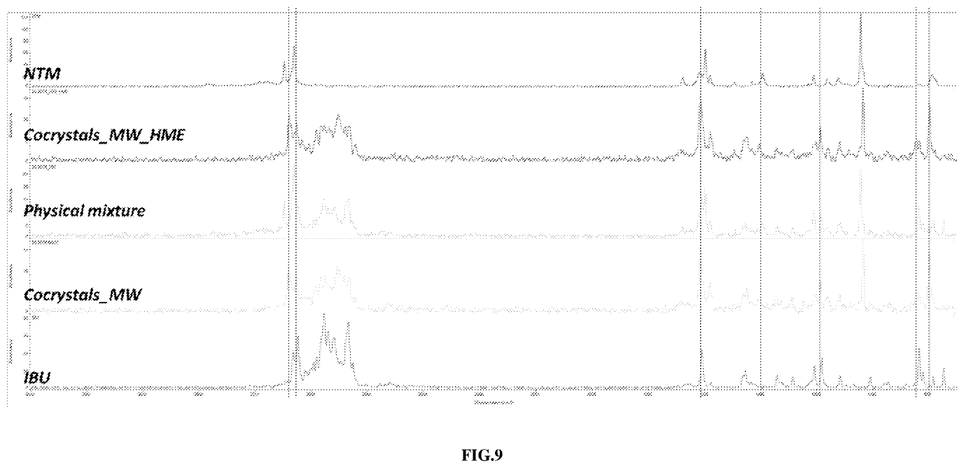

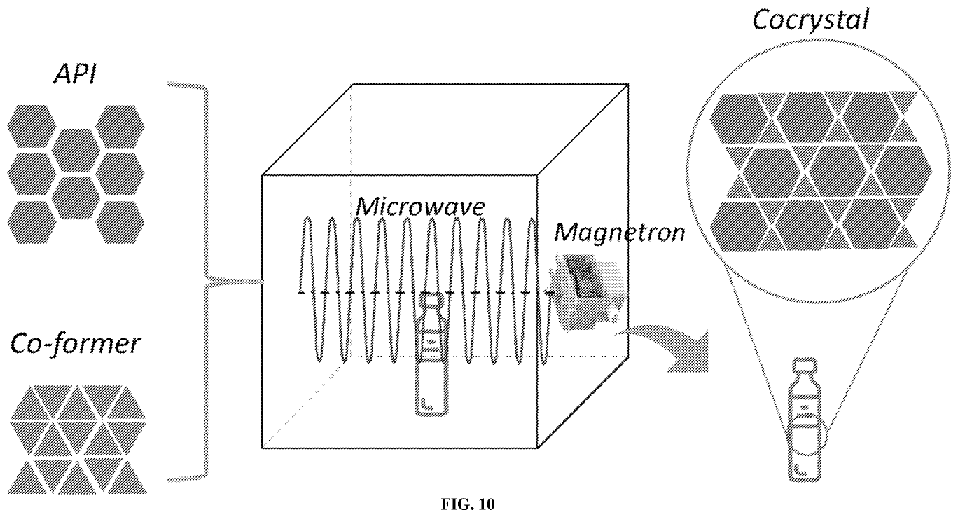

As shown in FIG. 10, 5 g of physically mixed powders was stored in a 20 ml vial then put in the middle of a Panasonic NE-1054F microwave oven, which generates the high-frequency microwave (2450 MHz). The heating power was 1000 W, the frequency was 2450 MHz and the wavelength was around 12.24 cm. The sample were heated until melt then take out from the oven and cool down to room temperature (25° C.). An infrared thermal detector was used to determine the final temperature of the heated tablets, and the heating time was recorded.

II. Characterization of the Cocrystals

The produced cocrystals were collected and characterized using various solid states characterization techniques including DSC, PLM, FT-IR, and Raman spectroscopy.

i. PLM

As shown in FIG. 11, the physical mixtures of each formulation were shown two different forms of crystals under the polarized light, on the contrary, the cocrystals showed single formed cocrystals. Further characterizations were conducted formation of cocrystals.

ii. DSC

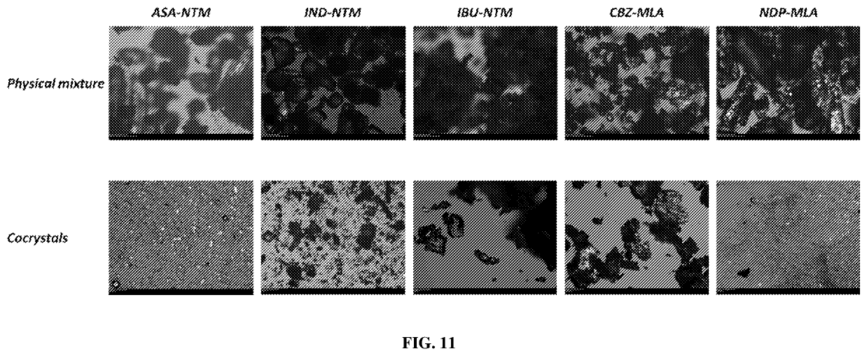

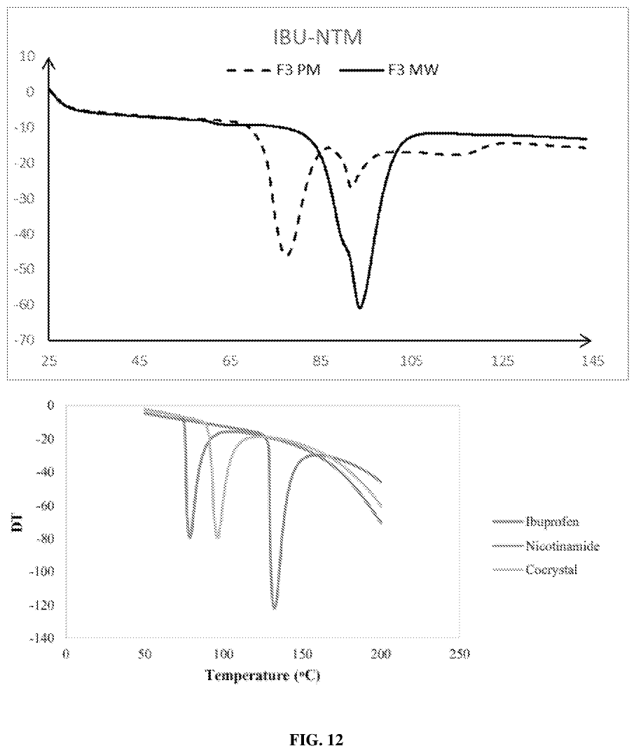

The IBU-NTM formulation was used as an example here. As shown in FIG. 12, left panel, the physical mixture shows a melting peak of IBU around 77.8° C., but NTM melting peak around 92.1° C. which might due to the NTM dissolve into the molten IBU. The cocrystal only showed a single melting peak of 93.7° C., which matched the literature work (FIG. 12, right panel) IBU-NTM cocrystals manufactured via solvent methods.

iii. XRD

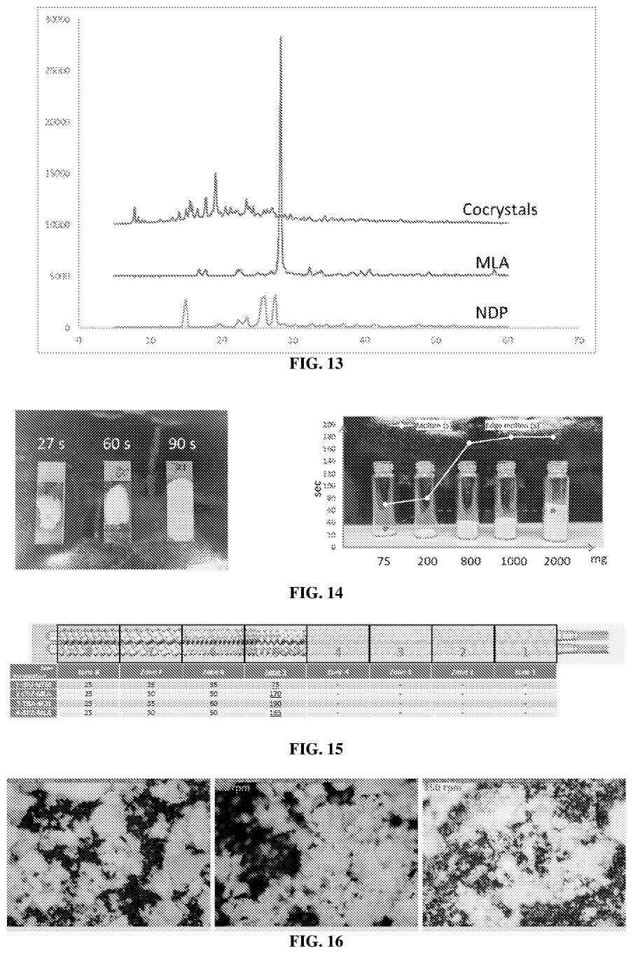

The NDP-MLA formulation was used as an example here. As shown in FIG. 13, the cocrystals new peaks at 2 theta range of 7.84, 13.2, 15.56, and 19.16, which was not seeing from neither NDP nor MLA crystals, which indicates the formation of the cocrystals.

III. Study of the Dielectric Heating Process

TABLE 2
The heating time and final temperature of each formulation.

| | | Molten | Edge molten | Final T |
|---|---|---|---|---|
| API (MP) | Coformer (MP) | (s) | (s) | (° C.) |
| ASA (135° C.) | NTM (130° C.) | 420 | NA | 132.7 |
| IND (158° C.) | NTM (130° C.) | 370 | 310 | 136.7 |
| IBU (80° C.) | NTM (130° C.) | 420 | NA | 161.8 |
| CBZ (190° C.) | MLA (130° C.) | 315 | 210 | 159.4 |
| NDP (173° C.) | MLA (130° C.) | 630 | 270 | 161.8 |

As shown in Table 2, the heating time for 5 g samples was neither positive nor negative proportional to the melting point of each of the components, which indicates the melting points might not be significant factors for the heating time. Additionally, the heating time might be affected by the density, the crystal size, or the sample positions. Since the heating endpoints were determined via the naked eye, so the final temperature might not be an adequate indicator to evaluate the heating process, but the data shown in Table 2 indicate that dielectric heating should bring the temperature at least higher than any one of the components in the formulation.

The IBU-NTM formulation was used for further study of the dielectric heating process. As shown in the FIG. 13, 72 mg, 200 mg, and 721 mg samples were put on the glass slides and spread as a thin layer, then put in the bottom middle of the oven, the heating time for each slide is around 27 s, 60 s, and 90 s, respectively. Due to the dielectric heating was using a microwave, and the wavelength is around 12.24 cm, so the heating power in the horizontal directions were not uniform. For 72 mg and 200 mg samples, all the particles almost melt at the same time, but for 721 mg samples, the edge starts melting around 60 s and completely melts around 90 s. The result showed that the amounts of samples and the position of the samples significantly affected the heating efficiency and uniformity.

Another study was tried by loading 75, 200, 800, 1000, and 2000 mg samples in a 5 ml vial, then heating inside the oven. For such small amounts of samples, the edges start melting almost at the same time which indicates the heating is efficient, but the amounts of samples will affect overall heating times. See FIG. 14.

### Example 3— Further Optimizations and Development of Formulations

### A. Formulations

Additional cocrystal formulations prepared using a dielectric heating combined with HME process is listed in Table 3. The physicochemical properties were characterized via a range of solid-state analyses as described below.

TABLE 3
Cocrystals compositions comprising API and co-formers

| | API | Co-former | Molar ratio |
|---|---|---|---|
| 1 | Ibuprofen (IBU) | Nicotinamide (NTM) | 1:1 |
| 2 | Carbamazepine (CBZ) | Maleic acid (MLA) | 1:1 |
| 3 | Tranilast (TRA) | Saccharin (SCH) | 1:1 |
| 4 | Aripiprazole (APZ) | Maleic acid (MLA) | 1:1 |

### B. Ibuprofen-Nicotinamide

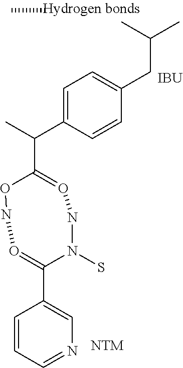

The IBU-NTM physical mixtures were premixed and loaded into the vessel and placed in the microwave system at room temperature, and then the dielectric heating was set at different powers to investigate the heating process (details below). The molten materials were then fed into the extruder, and the extruder was processed at ambient temperature (only zone 5-feeding zone were set at 75° C.), and cocrystal particles were discharged from zone 8. As shown above, the IBU and NTM can form cocrystals by forming a potential hydrogen bond between the amine and carboxyl groups, confirmed via solid states analysis.

i. The Heating Time

The heating time can be defined as the time required for the loaded solid materials to completely melt. As shown here, the heating time is affected by the total amount sample loaded and the respective energy applied or heating power (W).

As shown in Table 4, the microwave power was set at 800 W, and it was interesting to observe that regardless of the sample size, the amount of time needed to reach 80° C. was almost at the same (around 5 min). However, the actual time required for the samples to completely melt increased with the increase of the sample size. This indicates that the higher the mass of the samples loaded, the longer the heating time.

TABLE 4
The time needed to reach 80° C. and completely
melt the samples for different amounts of loading.

| Weight (g) | Complete Molten | Time to Reach 80° C. |
|---|---|---|
| 50 | ~8 min 15 s | 4 min 45 s |
| 100 | ~11 min 45 s | 5 min 15 s |
| 500 | ~16 min 30 s | 5 min 30 s |

As shown in Table 5, the heating time was significantly affected by the power inputs, where the 300 W is not high enough to reach 80° C. in 5 min, and any power greater than 600 W can achieve the set temperature at 5 min. Additionally, the greater power inputs will lead to a short heating time.

TABLE 5
The time needed to reach 80° C. and completely
melt the samples under different heating powers.

| Power (W) | Complete Molten | Time to Reach 80° C. |
|---|---|---|
| 300 | >15 min | N/A |
| 600 | 12 min 30 s | 5 min |
| 800 | 8 min 15 s | 5 min |
| 1000 | 5 min 45 s | 5 min |

ii. Extrusion Process and Determination of Residence Time

The extrusion process was dominated by the screw speed and feeding rates, which varied from minutes to hours. Additionally, the residence time can affect the formation of the cocrystals and the products' quality, morphology, and other characteristics. Three extrusion conditions were tried at the same temperature settings where feeding rate-screw speed was set at 6 g/min-50 rpm, 9 g/min-75 rpm, and 18 g/min-150 rpm. The residence time of each condition was 3.78 min, 2.80 min, and 1.22 min, and output 4.98 g/min, 8.31 g/min, and 17.52 g/min, respectively.

The process parameters and results are listed in Table 6. The extrusion of the IBU-NTM combinations is smooth where the torque was around 5.0 N/m (2.5% of maximum torque loading), which might be because of the complete melting of IBU and NTM in the mixing zone. The higher rpm results in a shorter residence time. The yield of the continuous extrusion can be as high as ˜1 kg/h using the process parameters used herein.

TABLE 6
Process results from extrusion of
IBU-NTM cocrystals at different conditions.

| Batch | 6 g/min-50 rpm | | 9 g/min-75 rpm | | 18 g/min-150 rpm | |
|---|---|---|---|---|---|---|
| Torque | 5.0 | N/m | 5.0 | N/m | 4.9 | N/m |
| Residence time | 3 min 47 s | | 2 min 48 s | | 1 min 12 s | |
| Output | 0.2988 | kg/h | 0.4968 | kg/h | 1.0512 | kg/h |
| D50 | 123 | μm | 117 | μm | 98 | μm |
| AOR | 23.5° | | 24.3° | | 24.9° | |
| Tap density | 53.73 | g/ml | 58.06 | g/ml | 62.07 | g/ml |

The IBU-NTM cocrystals obtained from the extrusion process are mainly granules rather than big agglomerates or lumps (FIG. 16) which are often found in traditional solvent free extrusion process, where the D50 of granules decreased with increasing screw speed. Without wishing to be bound by any theory, it is believed that the higher mechanical force or workloads to the downsizing of the granules.

The flowability of the cocrystals was identical, where the screw speed showed an insignificant effect on the Angle of Repose (AOR). The tap density of the cocrystals depended on the particle sizes, which were significantly affected by the screw speed.

The 6 g/min-50 rpm batch was repeated three times for reproducibility assessment. As shown in Table 7, the continuous extrusion of the cocrystals process showed adequate reproducibility, where most of the three batches' variation was within 5%. The variation of D50 is 12.62%, which is still within an acceptable range for oral dosage forms, and this might be due to the fact that the process condition and the sample collection and measuring procedure will affect the results.

TABLE 7
Reproducibility study of the continuous cocrystal extrusion process.

| 6 g/min-50 rpm | Batch 1 | | Batch 2 | | Batch 3 | | Average | | Variation |
|---|---|---|---|---|---|---|---|---|---|
| Residence time | 3 min 42 s | | 3 min 53 s | | 3 min 46 s | | 3 min 47 s | | 2.45% |
| Output | 0.2802 | kg/h | 0.3055 | kg/h | 0.3107 | kg/h | 0.2988 | kg/h | 5.46% |
| D50 | 138 | μm | 124 | μm | 107 | μm | 123 | μm | 12.62% |

| AOR | 23.4° | 23.4° | 23.6° | 23.5° | | 0.49% |
|---|---|---|---|---|---|---|
| Tap density | 52.98 | 54.07 | 54.14 | 53.73 | g/ml | 1.21 |

iii. Identification

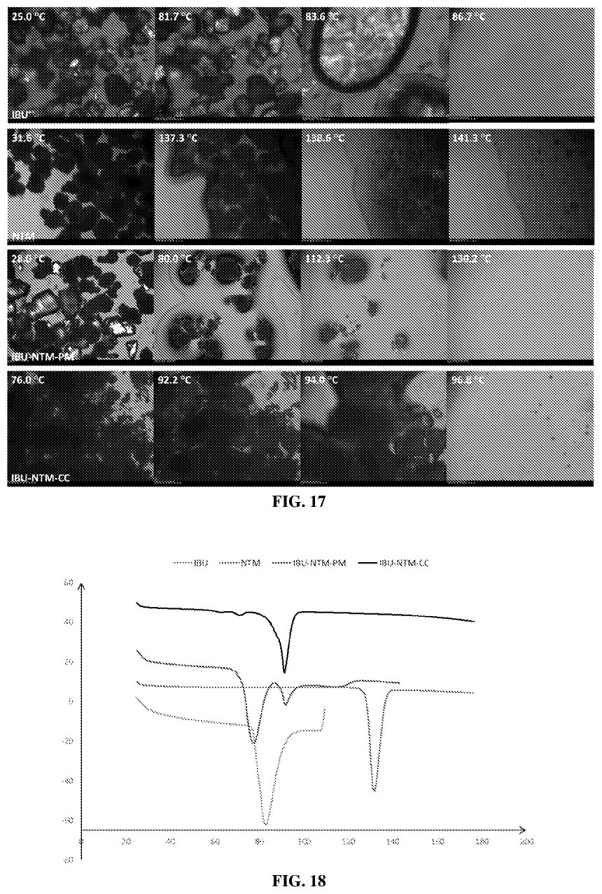

PLM figures (FIG. 17) showed that IBU and NTM melt at around 84° C. and 139° C., respectively. The physical mixture of the IBU-NTM starts melting at around 80° C., which is because of IBU, while NTM melts or dissolves into the molten IBU before reaching 130° C. This is mainly because of the interaction between the two molecules' function groups, which results in the adequate miscibility of two ingredients. A small fraction starts to melt at around 90° C., which is mainly due to the existence of free IBU or smaller particles. The cocrystals showed a single step of melting at 94° C. instead of two steps shown in the physical mixtures, which potentially proved the formation of a single-phase crystalline entity such as cocrystals.

DSC figures (FIG. 18) can cross-verify the observation from the PLM figures, where IBU and NTM have a melting peak of around 82.87° C. and 131.34° C., respectively. Furthermore, two isolated melting peaks can be observed in the physical mixture curve, where the first peak corresponds to the melting of IBU and the second one indicates the melting of NTM. The thermal transition for cocrystal formulation showed a melting peak of 91.29° C. corresponding to the melting of the IBU-NTM cocrystal.

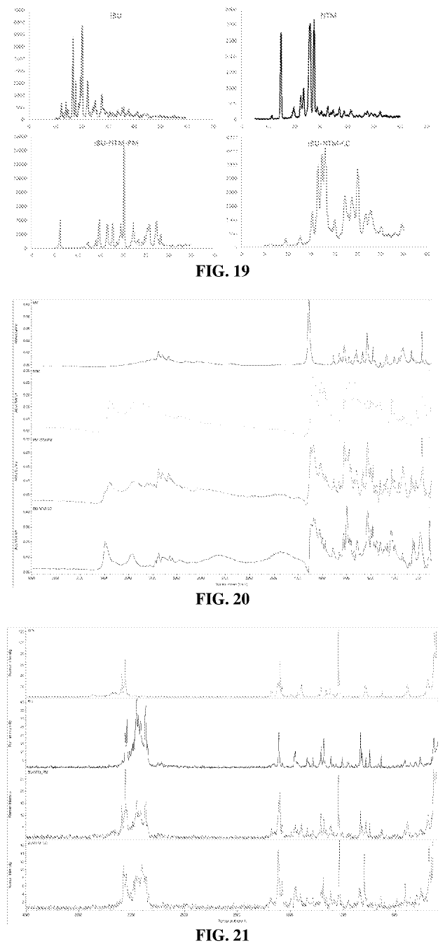

PXRD (FIG. 19) also proved the formation of the IBU-NTM cocrystals where the IBU showed characteristic peaks around 20 of 16.80, 17.68, 19.48, 20.24, 22.32, and 27.68°, and NTM exhibited diffraction peaks at around 20 of 15.00, 22.30, 23.12, and 27.50°. The cocrystals showed characteristic peaks at around 20 of 16.50, 17.36, 18.10, 25.12, and 28.12° 2θ positons. An additional new peak at ˜10° 2θ position is evident for the cocrystal formulation. This particular peak is not present in any of the bulk components nor in the physical mixture which indicates the formation of new crystal forms other than the IBU or NTM.

FTIR (FIG. 20) showed the intermolecular interactions of the cocrystals. The carboxyl group of IBU can be identified at around wavenumbers of 1718 and 930 cm⁻¹, while the amine group of NTM can be identified at wavenumbers of 1540-1450 cm⁻¹. The existence of the hydrogen bond in cocrystals results in the shift of the abovementioned peaks to the lower wavenumbers.

Raman (FIG. 21) showed the intramolecular movements of the cocrystals. The N—H movement of amine can be identified at the Raman shift of 1049 cm⁻¹ in NTM and physical mixtures, while it shifts to lower wavenumbers (1035 cm⁻¹) in the IBU-NTM cocrystals. The asymmetrical stretching can be identified at around 3035-3110 cm⁻¹ in IBU, NTM, and physical mixtures, while it is broadened to 3003-3129 cm⁻¹ in the IBU-NTM cocrystals because of hydrogen bonding.

### C. Carbamazepine-Maleic Acid (1:1)

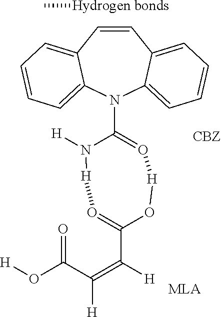

About 70 g of 1:1 molar ratio CBZ-MLA were premixed and loaded into the vessel and placed in the microwave system at room temperature, where the dielectric heating system was set at 800 W to heat from room temperature to 184° C. within 5 min and then held at 184° C. for 30 min. The samples were utterly molten after dielectric heating and then were fed into the extruder. The extruder was processed at ambient temperature (only zone 5-feeding zone were set at 170° C.). Unlike the IBU-NTM cocrystals, the CBZ-MLA cocrystal was discharged from zone 8 as semi-solids that haven't been completely solidified. The samples were collected and stored in a desiccator at ambient temperature prior to milling them into powders. As shown above, the CBZ and MLA can form cocrystals by forming a hydrogen bond between the amide and carboxyl groups, confirmed via solid states analysis.

i. Identification

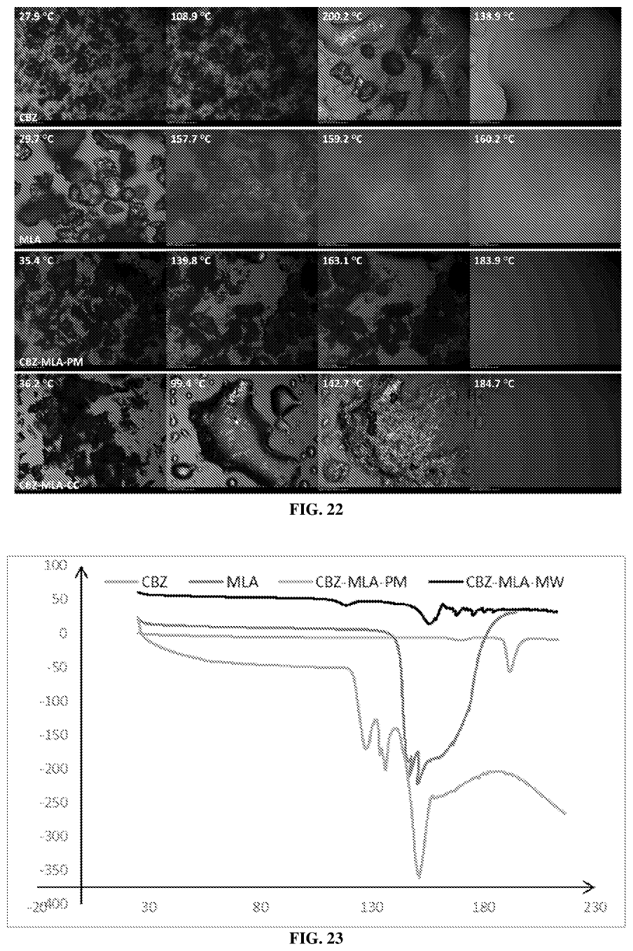

PLM figures (FIG. 22) showed that CBZ and MLA melt at around 200° C. and 160° C., respectively. The physical mixture of the CBZ and MLA starts melting around 140° C., which is because the MLA melts or dissolves into the molten CBZ before reaching 184° C. This is mainly because of the interaction between the two molecules' function groups, which results in the adequate miscibility of two ingredients. The cocrystals showed an interesting two-step of melting: the cocrystal almost melts before reaching 100° C. However, the CBZ transformed into a different polymorph (form I) around 143° C., and then it melt entirely at around 184° C. The melting behavior potentially proved the formation of a single-phase crystalline entity such as cocrystals.

DSC figures (FIG. 23) can cross-verify the observation from the PLM figures, where CBZ has a melting peak around 191.07° C., while the MLA has a broad endothermic peak at around 130.97° C. And multiple isolated melting peaks can be observed in the physical mixture curve, where the peaks before reaching 140° C. correspond to the eutectic system of CBZ and MLA. The cocrystal's thermal transition showed a smaller peak at around 119.17° C. which matches the observation from the PLM (most of the particles melts around 100° C.). Additionally, the cocrystal also showed another melting peak at around 157.71° C.

PXRD (FIG. 24) also proved the formation of the CBZ-MLA cocrystals where the CBZ (form III) showed characteristic peaks around 2θ of 14.9, 15.2, 15.8, 27.2, 27.5, and 32.0°, and MLA showed a strong peak around 2θ of 28.26°. The cocrystals showed characteristic peaks around 20 of 6.76, 8.72, 20.28, 22.36, and three peaks around 26.92, 27.52, and 28.04° which are different from the CBZ, MLA, and physical mixtures. Two additional new peaks between 7-9° 2θ positions is evident for the cocrystal formulation. These particular peaks are not present in any of the bulk components nor in the physical mixture which indicates the formation of new crystal forms other than the CBZ or MLA.

FTIR (FIG. 25) showed the intermolecular interactions of the cocrystals. There are two carboxyl groups of MLA and can be identified at around wavenumbers of 1718 and 930 cm⁻¹, while the amine group of CBZ can be identified at around wavenumbers of 1675 cm⁻¹ (N—H bend) and 1249 cm⁻¹ (C—N stretch). The existence of the hydrogen bond (around 952 cm⁻¹) in cocrystals results in the shift of the abovementioned peaks to the lower wavenumbers.

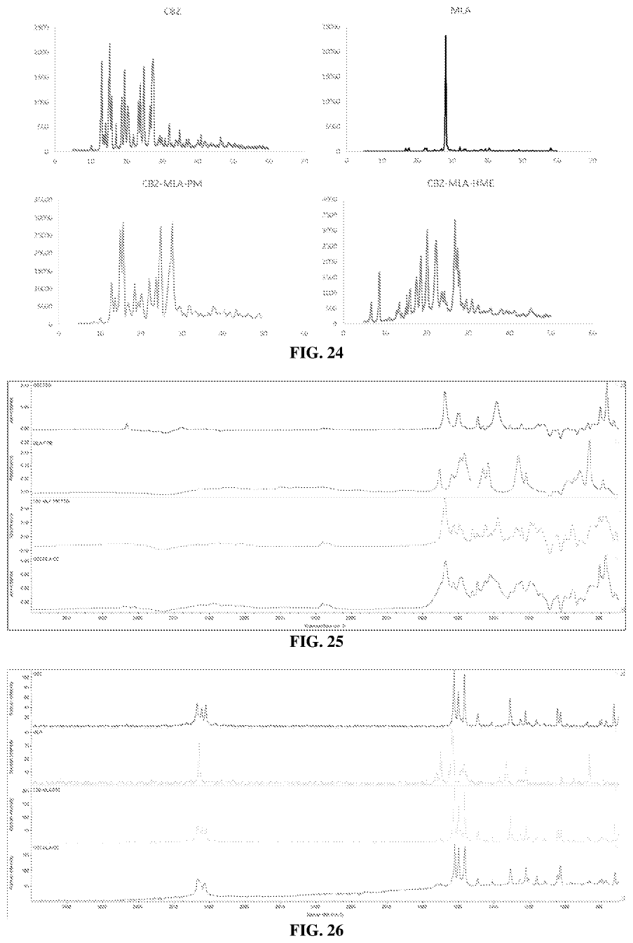

Raman (FIG. 26) showed the intramolecular movements of the cocrystals. The N—H movement of amine can be identified at the Raman shift of 1049 cm⁻¹ in CBZ and physical mixtures, while it shifts to lower wavenumbers (1035 cm⁻¹) in the CBZ-MLA cocrystals. The asymmetrical stretching can be identified around 3019-3070 cm⁻¹ in CBZ, MLA, and physical mixtures, while it is shifted to 3026-3075 cm⁻¹ in the CBZ-MLA cocrystals because of hydrogen bonding.

### D. Tranilast-Saccharin (1:1)

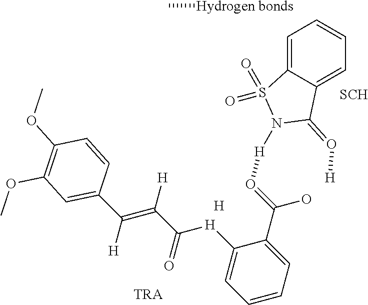

About 40 g of 1:1 molar ratio TRA-SCH were premixed and load into the vessel and placed in the microwave system at room temperature, where the dielectric heating system was set at 800 W to heat from room temperature to 210° C. within 5 min and then held at 210° C. for 5 min. The samples were completely molten after dielectric heating, and then were fed into the extruder. The extruder was processed at ambient temperature (only zone 5-feeding zone were set at 190° C.). The samples were collected and stored in a desiccator in ambient temperature prior to milling them into powders. As shown above, the TRA and SCH can form cocrystals by forming a hydrogen bond between the amide and carboxyl groups, confirmed via solid states analysis.

i. Identification

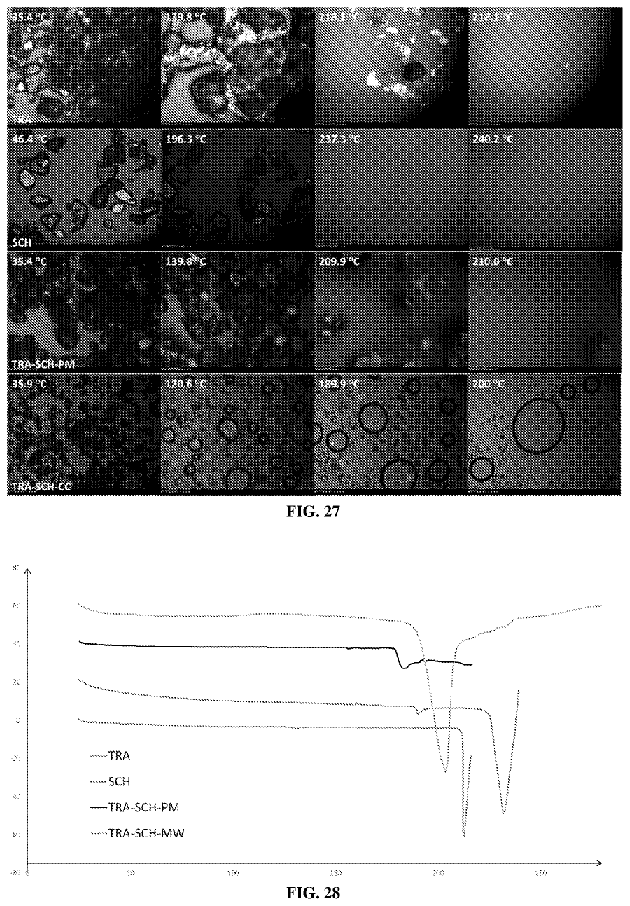

PLM figures (FIG. 27) showed that TRA and SCH melt at around 218.1° C. and 240.2° C., respectively. The physical mixture of the TRA and SCH starts melting around 209.9° C., which is because of the TRA-SCH formed a eutectic system which has a eutectic point that is lower than any of the composition ingredients. The cocrystals showed an interesting melting behavior at higher temperature than the individual components: the cocrystal starts melting at around 120° C. which completes at around 200° C. The melting behavior potentially proves the formation of a single-phase crystalline entity such as cocrystals.

DSC figures (FIG. 28) can cross-verify the observation from the PLM figures, where TRA has a melting peak at around 212.61° C., while the SCH exhibits a broad endothermic peak at around 231.97° C. An attenuated melting peak can be observed for the physical mixture, where the peak at 183.49° C. corresponds to the eutectic system of TRA and SCH. The cocrystals thermal transition showed a smaller peak at around 203.99° C. which is in accordance with the observation from the PLM.

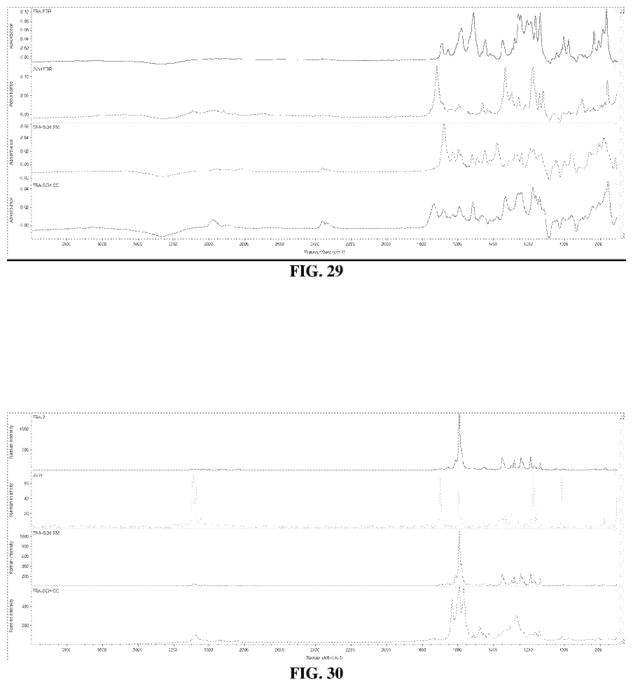

FTIR (FIG. 29) showed the intermolecular interactions of the cocrystals. There is a carboxyl groups of TRA and can be identified at around wavenumbers of 1691 and 939 cm⁻¹, while the amide group of SCH can be identified at around wavenumbers of 1453 cm⁻¹ (C—N stretch) and 1590 cm⁻¹ (N—H in plane). The existence of the hydrogen bond (around 953 cm⁻¹) in cocrystals results in the shift of the C═O peaks from 1714 cm⁻¹ to 1740 cm⁻¹.

Raman (FIG. 30) showed the intramolecular movements of the cocrystals. The C═O mixed with N—H deformation of amide can be identified at the Raman shift of 1602 cm⁻¹ in TRA, SCH, physical mixtures, and cocrystals. In the physical mixtures, it seems the TRA is more sensitive to the Raman signals and blocked the signal from SCH where it is showing almost no characteristic peaks from SCH. In facts, the new peaks at wavenumbers of 1640 and 1560 cm⁻¹ (interaction between carboxyl and amide groups) in the TRA-SCH cocrystals may prove the formation of new solid phase of crystals.

### E. Aripiprazole-Maleic Acid (1:1)

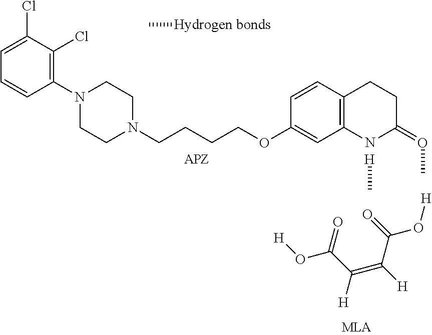

Approximately, 40 g of 1:1 molar ratio APZ-MLA were premixed and load into the vessel and placed in the microwave system at room temperature, where the system was set at 800 W to heat from room temperature to 150° C. within 5 min and then held at 150° C. for additional 5 min. The samples were completely molten after dielectric heating, and then were fed into the extruder. The extruder was processed at ambient temperature (only zone 5-feeding zone were set at 165° C.). The samples were collected and stored in a desiccator in ambient temperature which were then subject to milling into powders. As shown above, the APZ and MLA can form cocrystals by forming a hydrogen bond between the amide (from lactam) and carboxyl groups, confirmed via solid states analysis.

i. Identification

PLM figures (FIG. 31) showed that APZ and MLA melt at around 152.6° C. and 160.2° C., respectively. The physical mixture of the TRA and SCH starts melting at around 106.4° C., which is because TRA-SCH has a eutectic point that is lower than any of the composition ingredients. The cocrystals showed an interesting melting behavior: the cocrystal starts melting at around 183.4° C., which is higher than both the melting points of each ingredients. The melting behavior potentially proved the formation of a single-phase crystalline entity such as cocrystals.

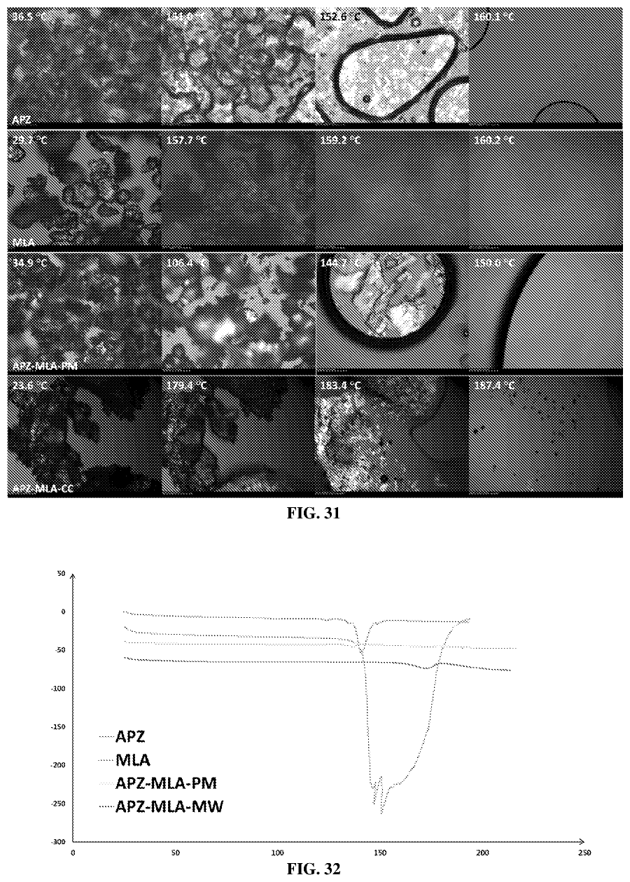

DSC figures (FIG. 32) can cross-verify the observation from the PLM figures, where APZ has a melting peak at around 141.59° C., while the MLA has a broad endothermic peak at around 130.97° C. The cocrystals showed an attenuated peak around 172.99° C. which is matching the observation from the PLM (most part of particles melts around 183.4° C. and completely melts around 187.4° C.).

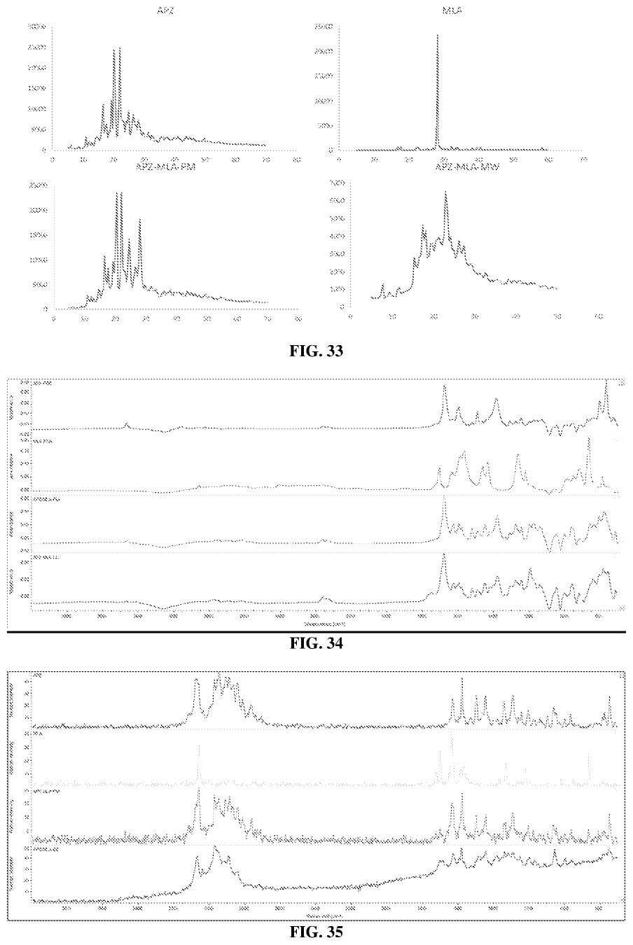

PXRD (FIG. 33) also proved the formation of the APZ-MLA cocrystals where the APZ showed characteristic peaks around 20 of 16.6, 19.4, 20.2, 22.2, 25.0, and 28.2°, and MLA showed a strong peak around 20 of 28.26°. The cocrystals showed a characteristic peak around 20 of 17.4, 18.2, 19.4, 21.4, and 23.0°, which different from the APZ, MLA, and physical mixtures. Additional new peaks at about 6.5-9 2θ position are evident for the cocrystal formulation. These particular peaks are not present in any of the bulk components nor in the physical mixture which indicates the formation of new crystal forms other than the APZ or MLA.

FTIR (FIG. 34) showed the intermolecular interactions of the cocrystals. There are two carboxyl groups of MLA and can be identified at around wavenumbers of 1718 and 930 cm⁻¹, while the cyclic amide group from the lactam in APZ can be identified at wavenumbers of 1670 cm⁻¹. The existence of the hydrogen bond in cocrystals results in the shift of the abovementioned peaks to the lower wavenumbers.

Raman (FIG. 35) showed the intramolecular movements of the cocrystals. The N—H movement of amine can be identified at the Raman band around 1575 cm⁻¹ in APZ and physical mixtures, while it shifts to the wavenumbers of 1079 cm⁻¹ in the APZ-MLA cocrystals. The asymmetrical stretching can be identified around 3019-3148 cm⁻¹ in APZ, MLA, and physical mixtures, while it is shifted to 3026-3108 cm⁻¹ in the APZ-MLA cocrystals because of hydrogen bonding.

### Example 4—Methods and Materials

### A. Dielectric Heating Power and Time

The heating time was studied via loading different amounts of materials into the vessel of the microwave system, where the heating power was kept constant at 800 W. Approximately 50, 100, and 500 g of IBU-NTM physical mixtures were loaded into the microwave sample vessel. The heating power was studied via loading a constant amount of the samples such as 50 g IBU-NTM physical mixtures into the vessel and then by subjecting it to heating at varying heating power such as at 300, 600, 800, and 1000 W. The heating profiles were set as heating samples from ambient temperature (25° C.) to 80° C. for the first 5 min and then holding at 80° C. for 30 min.

### B. Feeding Rates and Screw Speed

For formulations containing IBU-NTM combination, screw speed and feeding rate were studied. Three extrusion conditions were tried at the same temperature settings where feeding rate-screw speed was set at 6 g/min-50 rpm, 9 g/min-75 rpm, and 18 g/min-150 rpm.

### C. Process Quality

i. Reproducibility

Reproducibility studies were conducted with formulation ASA-NTM, where the 6 g/min-50 rpm condition was repeated three times.

ii. Measurement of the Residence Time

Residence times were recorded during the extrusion process when it reached a steady state and where the torque changes were within 5% variations. Around 100 mg of tracer dye (Rhodamine B) were added in zone 1 during the extrusion, and the time needed to observe a steady color change at zone 8 was recorded as the residence time.

### D. DSC

A DSC Q20 equipment (TA® instruments, Delaware, USA) was used for the DSC analysis. Approximately 5-10 mg of pure API, co-former, physical mixtures, and extruded cocrystals were sealed in the standard aluminum pan and lids and ramped from 25 to complete melting temperature (depending on the samples) at a rate of 20° C./min. In all DSC experiments, ultra-purified nitrogen was used as the purge gas at a 50 mL/min flow rate. The data were collected and plotted as a plot of temperature (° C.) versus reverse heat flow (mW) using Microsoft Excel (Version 2007).

### E. Powder X-Ray Diffraction (PXRD)

The solid state of pure API, co-former, physical mixtures, and extruded cocrystals were investigated using a benchtop PXRD instrument (MiniFlex, Rigaku Corporation, Tokyo, Japan). Briefly, the samples were scanned from a 2θ angle of 5 to 60 degrees, with a scan speed of 2 degrees/minute, scan step of 0.02 degrees, and the resultant scan resolution of 0.0025. The voltage was set at 45V, and the current was set at 15 mA during the scan process. The data were collected and plotted as a stacked plot of 2θ versus intensity using Excel software (Version 2007).

### F. Hot-Stage Polarized Light Microscopy (PLM)

An Olympus BX53 polarizing photomicroscope (Olympus America Inc., Webster, TX, USA) equipped with Bertrand Lens was used to analyze the crystallinity of the pure API, co-former, physical mixtures, and extruded cocrystals. The samples were spread out evenly onto a glass slide. A coverslip was used to press and spread the samples as monolayer particles. The slide was placed on the microscope stage. All samples were observed under 10× magnification for birefringence property in crystalline substances. A QICAM Fast 1394 digital camera (QImaging, BC, Canada) with a 530 nm compensator (U-TP530, Olympus® corporation, Shinjuku City, Tokyo, Japan) was used to capture the images.

### G. Fourier-Transform Infrared (FTIR) Spectroscopy

The pure API, co-former, physical mixtures, and extruded cocrystals were investigated using an Antaris analyzer with Nicolet iS50 series spectrophotometer (Thermal Fisher Scientific, Madison, WI, USA). Samples were positioned onto the face of the diamond crystal of the ATR unit, and the tip of the micrometer clamp was compressed onto the particles to allow adequate contact to get a characteristic spectrum. Backgrounds were collected using 32 scans for each 4 h during the FTIR measurements, while the spectrum of the sample was by scanning the specimens 32 times over a 750-4000 cm⁻¹ range at a resolution of 4 cm⁻¹ per sample.

### H. Raman Spectroscopy

The pure API, co-former, physical mixtures, and extruded cocrystals were investigated using an Antaris analyzer (Thermal Fisher Scientific, Madison, WI, USA) equipped with a Raman scanning unit. Samples were positioned onto a 9-well sample holder. The samples spectrum was by scanning the specimens 32 times over a 300-4000 cm⁻¹ range at a 4 cm⁻¹ per sample.

### I. Cocrystals Particle Size and Distribution

Images of the cocrystals were taken using Dino-Lite optical microscopy. Particle size was measured using DinoCapture 2.0 (version 1.5.43), where particles were circled using 3-points circles. Approximately 5 different figures for replicates of each cocrystal were taken, and diameter d was recorded then exported to Excel for further analysis.

### J. Angle of Repose

The material was poured through a funnel to form a cone. Pouring was stopped when the pile reaches a predetermined height or the base a predetermined width. The AOR was calculated by dividing the cone height by half the width of the base of the cone. The inverse tangent of this ratio is the angle of repose.

### K. Tapped Density

Tapped density of cocrystals was obtained as the ratio of the mass of the cocrystals to the volume occupied by the powder after it has been tapped for a defined period. The tapping period is defined as the volume changing <5%, and for all the formulations, it's around 100 taps.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit, and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope, and concept of the disclosure as defined by the appended claims.

### REFERENCES

The following references, to the extent that they provide exemplary procedural or other details supplementary to those set forth herein, are specifically incorporated herein by reference.
U.S. Pat. No. 8,486,423
U.S. Pat. No. 9,339,440
Abourahma et al., CrystEngComm, 13:6442-50, 2011.
Agrawal and Patel, J. Adv. Pharm. Technol. Res., 2:81, 2011.
Ahuja et al., CrystEngComm., 22:1381-94, 2020.
Bergese et al., Mater. Sci. Eng. C., 23:791-5, 2003.
Crowley et al., Drug Dev. Ind. Pharm., 909-26, 2007.
Daurio et al., Faraday Discuss., 170:235-49, 2014.
DiNunzio et al., Drug Development and Industrial Pharmacy, 36(9):1064-78, 2010d.
DiNunzio et al., European Journal of Pharmaceutical Sciences, 40(3):179-87, 2010c.
DiNunzio et al., European Journal of Pharmaceutics and Biopharmaceutics, 74(2):340-51, 2010a.
DiNunzio et al., Journal of Pharmaceutical Sciences, 99(3):1239-53, 2010b.
Elder et al., Int. J. Pharm., 88-100, 2013.
Gao and Morozowich, Expert Opin. Drug Deliv., 97-110, 2006.
Heimbach et al., Prodrugs Challenges Reward. Part 1, New York, NY: Springer New York; 2007 p. 157-215.
Kappe, Chem. Soc. Rev., 37:1127-39, 2008.
Kappe, Angew. Chemie—Int. Ed., 6250-84, 2004.
Karki et al., Mol. Pharm., 4:347-54, 2007.
Kelly et al., Int. J. Pharm., 426:15-20, 2012.
Kerč et al., Alternative Solvent-Free Preparation Methods for Felodipine Surface Solid Dispersions, 24:359-63, 1998.
Lang et al., Mol Pharm., 11(1):186-96, 2014b.
Lang et al., J Drug Deliv Sci Tec., 24(2):205-11, 2014a.
Maheshwari et al., Indian J. Pharm. Sci., 69:822-4, 2007.
Manin et al., Eur. J. Pharm. Sci., 65:56-64, 2014.
Moneghini et al., J. Drug Deliv. Sci. Technol. 18:327-33, 2008.
Pagire et al., CrystEngComm, 15:3705-10, 2013.
Passerini et al., Eur. J. Pharm. Sci., 15:71-8, 2002.
Paulekuhn et al., J. Med. Chem., 50:6665-72, 2007.
Pouton Eur. J. Pharm. Sci., 29:278-87, 2006.
Rosen and Kunjappu Surfactants and interfacial phenomena, John Wiley & Sons; 2012.
Vinarov et al., Drug Dev. Ind. Pharm., 44:677-86, 2018.
Wen et al., J. Pharm. Biomed. Anal., 34:517-23, 2004.
Wiesbrock et al., Rapid Commun., 1739-64, 2004.
Yuliandra et al., Sci. Pharm., 86, 2018.
Zhang et al., Int. J. Pharm., 519:186-97, 2016.
Zhou et al., J. Mater. Process. Technol. 137:156-8, 2003.

What is claimed is:

1. A method of preparing a pharmaceutical composition comprising:
(A) obtaining a mixture of an active pharmaceutical ingredient (API) and a co-former;
(B) subjecting the mixture to dielectric heating to obtain a pharmaceutical composition;
wherein the pharmaceutical composition comprises at least 50% of the API and the co-former is present as a co-crystal.

2. The method of claim 1, wherein at least 80% of the API and the co-former is present as a co-crystal.

3. The method of either claim 1 or claim 2, wherein at least 90% of the API and the co-former is present as a co-crystal.

4. The method according to any one of claims 1-3, wherein at least 95% of the API and the co-former is present as a co-crystal.

5. The method according to any one of claims 1-4, wherein at least 98% of the API and the co-former is present as a co-crystal.

6. The method according to any one of claims 1-5, wherein at least 99% of the API and the co-former is present as a co-crystal.

7. The method according to any one of claims 1-6, wherein the dielectric heating comprises using a specific frequency electromagnetic radiation.

8. The method of claim 7, wherein the specific frequency electromagnetic radiation is a radio wave.

9. The method of claim 8, wherein the radio wave has a frequency from about 10 MHz to about 20 MHz.

10. The method of claim 7, wherein the specific frequency electromagnetic radiation is a microwave.

11. The method of claim 10, wherein the microwave has a frequency greater than 100 MHz.

12. The method of claim 11, wherein the microwave has a frequency from about 500 MHz to about 1,000 GHz.

13. The method of claim 12, wherein the microwave has a frequency from about 1000 MHz to about 100 GHz.

14. The method of claim 13, wherein the microwave has a frequency from about 1000 MHz to about 25 GHz.

15. The method of claim 14, wherein the microwave has a frequency from about 1000 MHz to about 10 GHz.

16. The method of claim 15, wherein the microwave has a frequency from about 1000 MHz to about 3000 MHz.

17. The method according to any one of claims 1-16, wherein the dielectric heating comprise a heating power.

18. The method of claim 17, wherein the heating power is from about 200 W to about 10 kW.

19. The method of claim 18, wherein the heating power is from about 500 W to about 5 kW.

20. The method of claim 19, wherein the heating power is from about 750 W to about 2 kW.

21. The method of claim 20, wherein the heating power is from about 800 W to about 1,500 W.

22. The method according to any one of claims 1-21, wherein the dielectric heating comprises using energy having a specific wavelength.

23. The method of claim 22, wherein the specific wavelength is greater than 1 mm.

24. The method of claim 23, wherein the specific wavelength is from about 1 mm to about 1 m.

25. The method of claim 24, wherein the specific wavelength is from about 3 mm to about 300 mm.

26. The method of claim 25, wherein the specific wavelength is from about 50 mm to about 200 mm.

27. The method of claim 26, wherein the specific wavelength is from about 100 mm to about 150 mm.

28. The method according to any one of claims 1-27 further comprising subjecting the mixture to a composition processing method.

29. The method of claim 28, wherein the composition processing method is performed contemporaneously with subjecting the mixture to dielectric heating.

30. The method of claim 28, wherein the composition processing method is performed after with subjecting the mixture to dielectric heating.

31. The method of claim 28, wherein the composition processing method is performed before with subjecting the mixture to dielectric heating.

32. The method according to any one of claims 28-31, wherein the composition processing method is extrusion, fluidized bed granulation, high shear granulation, propeller mixing, turbine mixing, high shear mixing, high pressure or ultrasonic homogenization.

33. The method of claim 32, wherein the composition processing method is extrusion.

34. The method of claim 33, wherein the extrusion is hot melt extrusion.

35. The method according to any one of claims 32-34, wherein the extrusion comprises heating the extrusion composition to a first temperature.

36. The method of claim 35, wherein the first temperature is from ambient temperature to a temperature less than the melting of either the co-former or the API.

37. The method of either claim 35 or claim 36, wherein the first temperature is from about 10° C. to about 250° C.

38. The method of claim 37, wherein the first temperature is from about 50° C. to about 150° C.

39. The method according to any one of claims 35-38, wherein the method comprises a second temperature.

40. The method of claim 39, wherein the second temperature is from about 10° C. to about 250° C.

41. The method of claim 40, wherein the second temperature is from about 10° C. to about 100° C.

42. The method according to any one of claims 32-41, wherein the extrusion method comprises a screw speed from about 10 rpm to about 400 rpm.

43. The method of claim 42, wherein the screw speed is form about 20 rpm to about 300 rpm.

44. The method of claim 43, wherein the screw speed is from about 25 rpm to about 200 rpm.

45. The method of claim 44, wherein the screw speed is 50 rpm, 75 rpm, 100 rpm, 150 rpm, or 200 rpm.

46. The method according to any one of claims 33-44, wherein the productivity or throughput of extrusion is about 100 g/hr to 2.5 kg/hr relative to a lab scale twin-screw extruder.

47. The method according to any one of claims 33-44, wherein the productivity of extrusion is about 250 g/hr to 2.0 kg/hr

48. The method according to any one of claims 33-44, wherein the productivity of extrusion is about 360 g/hr, 500 g/hr, 540 kg/hr, 1.08 kg/hr, 2.0 kg/hr, or 2.5 kg/hr.

49. The method according to any one of claims 1-48, wherein the API is a BCS Class II drug.

50. The method according to any one of claims 1-48, wherein the API is a BCS Class IV drug.

51. The method according to any one of claims 1-50, wherein the API is an API with a melting point of less than 250° C.

52. The method of claim 51, wherein the melting point is less than 200° C.

53. The method according to any one of claims 1-52, wherein the API is selected from anticancer agents, antiallergic agents, antifungal agents, psychiatric agents such as analgesics, consciousness level-altering agents such as anesthetic agents or hypnotics, nonsteroidal anti-inflammatory agents (NSAIDs), anthelmintics, antiacne agents, antianginal agents, antiarrhythmic agents, anti-asthma agents, antibacterial agents, anti-benign prostate hypertrophy agents, anticoagulants, antidepressants, antidiabetics, antiemetics, antiepileptics, antigout agents, antihypertensive agents, anti-inflammatory agents, antimalarials, antimigraine agents, antimuscarinic agents, antineoplastic agents, anti-obesity agents, antiosteoporosis agents, antiparkinsonian agents, antiproliferative agents, antiprotozoal agents, antithyroid agents, antitussive agent, anti-urinary incontinence agents, antiviral agents, anxiolytic agents, appetite suppressants, beta-blockers, cardiac inotropic agents, chemotherapeutic drugs, cognition enhancers, contraceptives, corticosteroids, Cox-2 inhibitors, diuretics, erectile dysfunction improvement agents, expectorants, gastrointestinal agents, histamine receptor antagonists, immunosuppressants, keratolytics, lipid regulating agents, leukotriene inhibitors, macrolides, muscle relaxants, neuroleptics, nutritional agents, opioid analgesics, protease inhibitors, and sedatives.

54. The method of claim 53, wherein the API is an antifungal agent, a psychiatric agent, an antiallergic agent, a chemotherapeutic drug, an antibiotic, or a nonsteroidal anti-inflammatory agent.

55. The method of claim 54, wherein the API is a chemotherapeutic drug.

56. The method of claim 54, wherein the API is an antibiotic.

57. The method of claim 54, wherein the API is a nonsteroidal anti-inflammatory agent.

58. The method of claim 57, wherein the API is ibuprofen or acetylsalicylic acid.

59. The method of claim 54, wherein the API is an antihypertensive agent.

60. The method of claim 57, wherein the API is nifedipine.

61. The method of claim 54, wherein the API is an antifungal agent.

62. The method of claim 57, wherein the API is indomethacin.

63. The method of claim 54, wherein the API is an antiepileptic.

64. The method of claim 57, wherein the API is carbamazepine.

65. The method of claim 54, wherein the API is a psychiatric agent.

66. The method of claim 65, wherein the API is aripiprazole.

67. The method of claim 54, wherein the API is an antiallergic agent.

68. The method of claim 67, wherein the API is tranilast.

69. The method according to any one of claims 1-64, wherein the co-former interacts with the API through one or more non-covalent interactions.

70. The method of claim 69, wherein the non-covalent interactions are ionic interactions, hydrogen bonding, halogen bonding, van der Waals forces, π-π interactions, or hydrophobic effects.

71. The method according to any one of claims 1-70, wherein the co-former and the API interact with two or more non-covalent interactions.

72. The method according to any one of claims 1-70, wherein the co-former is a compound which modifies the solubility of the API.

73. The method of claim 72, wherein the co-former is a compound which is sparingly soluble and modifies the solubility of the API.

74. The method of claim 72, wherein the co-former is a compound which is sensitive to the environment and modifies the solubility of the active pharmaceutical ingredient.

75. The method of claim 74, wherein the compound is sensitive to the pH of the environment.

76. The method of claim 74, wherein the compound is sensitive to the temperature of the environment.

77. The method according to any one of claims 1-76, wherein the co-former is a compound that has no therapeutic effect.

78. The method according to any one of claims 1-76, wherein the co-former is a second API.

79. The method of claim 78, wherein the second API is for the same disease or disorder as the first API.

80. The method of claim 78, wherein the second API is for a different disease or disorder as the first API.

81. The method according to any one of claims 1-80, wherein the co-former comprises one or more functional groups selected from amine, amide, a nitrogen containing heterocycle, carbonyl, carboxyl, hydroxyl, phenol, sulfone, sulfine, sulfinyl, sulfonyl, mercapto, and methyl thio.

82. The method of claim 81, wherein the functional group is a NH₂, OH, C(O), C(O)OH, SH, or a nitrogen containing heterocycle.

83. The method of claim 82, wherein the functional group is a nitrogen containing heterocycle, NH₂, OH, or SH.

84. The method according to any one of claims 1-83, wherein the co-former is a carboxylic acid.

85. The method of claim 84, wherein the co-former is malic acid.

86. The method according to any one of claims 1-83, wherein the co-former is a vitamin or a vitamin derivative.

87. The method of claim 86, wherein the co-former is nicotinamide.

88. The method according to any one of claims 1-83, wherein the co-former is a flavoring agent.

89. The method of claim 88, wherein the co-former is saccharin.

90. The method according to any one of claims 1-89, wherein the pKₐ of the active pharmaceutical ingredient and the pKₐ of the co-former have a pKₐ difference of less than 3.

91. The method of claim 90, wherein the pKₐ difference is less than 2.

92. The method of claim 91, wherein the pKₐ difference is less than 1.

93. The method of claim 92, wherein the pKₐ difference is less than 0.5.

94. The method according to any one of claims 1-93, wherein the method results in a compositions showing improved flowability or is able to obtain more co-crystals in the pharmaceutical composition relative to either dielectric heating or an extrusion process alone.

95. The method according to any one of claims 1-93, wherein the mixture further comprises an excipient.

96. The method according to any one of claims 1-95 further comprising one or more further formulation steps.

97. The method of claim 96, wherein the further formulation steps including milling or grinding.

98. The method of either claim 96 or claim 97, wherein the further formulation steps comprise tableting, filling a capsule, formulating an oral suspension, formulating a film, or additive manufacturing techniques.

99. The method of claim 98, wherein the additive manufacturing technique is vat photopolymerization, material jetting, binding jetting, powder-bed fusion, material extrusion, directed energy deposition, sheet lamination, fused deposition modeling, binder spraying, or selective laser sintering.

100. A pharmaceutical composition comprising:
(A) an active pharmaceutical ingredient (API);
(B) a co-former;
wherein at least 50% of the API and the co-former is present as a co-crystal; and the pharmaceutical composition has been subjected to dielectric heating.

101. The pharmaceutical composition of claim 100, wherein at least 80% of the API and the co-former is present as a co-crystal.

102. The pharmaceutical composition of either claim 100 or claim 101, wherein at least 90% of the API and the co-former is present as a co-crystal.

103. The pharmaceutical composition according to any one of claims 100-102, wherein at least 95% of the API and the co-former is present as a co-crystal.

104. The pharmaceutical composition according to any one of claims 100-103, wherein at least 98% of the API and the co-former is present as a co-crystal.

105. The pharmaceutical composition according to any one of claims 100-104, wherein at least 99% of the API and the co-former is present as a co-crystal.

106. The pharmaceutical compositions according to any one of claims 100-105, wherein the co-crystals are in a single phase.

107. The pharmaceutical composition according to any one of claims 100-105, wherein the pharmaceutical composition comprises a Carr's Index from about 5 to about 30.

108. The pharmaceutical composition according to any one of claims 100-105, wherein the pharmaceutical composition comprises a surface area of greater than 100 m²/g.

109. The pharmaceutical composition according to any one of claims 100-108, wherein the pharmaceutical composition comprises a mean or average particle size distribution is from about 25 μm to about 500 μm.

110. The pharmaceutical composition of claim 109, wherein the mean or average particle size distribution is from about 50 μm to about 250 μm.

111. The pharmaceutical composition according to any one of claims 100-110, wherein the pharmaceutical composition has a flowability as a function of angle of repose of greater than about 25.

112. The pharmaceutical composition according to any one of claims 100-111, wherein the pharmaceutical composition comprises a drug content uniformity is from about 95% to about 105%.

113. The pharmaceutical composition according to any one of claims 100-112, wherein the dielectric heating comprises using a specific frequency electromagnetic radiation.

114. The pharmaceutical composition of claim 113, wherein the specific frequency electromagnetic radiation is a radio wave.

115. The pharmaceutical composition of claim 114, wherein the radio wave has a frequency from about 10 MHz to about 20 MHz.

116. The pharmaceutical composition of claim 113, wherein the specific frequency electromagnetic radiation is a microwave.

117. The pharmaceutical composition of claim 116, wherein the microwave has a frequency greater than 100 MHz.

118. The pharmaceutical composition of claim 117, wherein the microwave has a frequency from about 500 MHz to about 1,000 GHz.

119. The pharmaceutical composition of claim 118, wherein the microwave has a frequency from about 1000 MHz to about 100 GHz.

120. The pharmaceutical composition of claim 119, wherein the microwave has a frequency from about 1000 MHz to about 25 GHz.

121. The pharmaceutical composition of claim 120, wherein the microwave has a frequency from about 1000 MHz to about 10 GHz.

122. The pharmaceutical composition of claim 121, wherein the microwave has a frequency from about 1000 MHz to about 3000 MHz.

123. The pharmaceutical composition according to any one of claims 100-122, wherein the dielectric heating comprise a heating power.

124. The pharmaceutical composition of claim 123, wherein the heating power is from about 200 W to about 10 kW.

125. The pharmaceutical composition of claim 124, wherein the heating power is from about 500 W to about 5 kW.

126. The pharmaceutical composition of claim 125, wherein the heating power is from about 750 W to about 2 kW.

127. The pharmaceutical composition of claim 126, wherein the heating power is from about 800 W to about 1,500 W.

128. The pharmaceutical composition according to any one of claims 100-127, wherein the dielectric heating comprises using energy having a specific wavelength.

129. The pharmaceutical composition of claim 128, wherein the specific wavelength is greater than 1 mm.

130. The pharmaceutical composition of claim 129, wherein the specific wavelength is from about 1 mm to about 1 m.

131. The pharmaceutical composition of claim 130, wherein the specific wavelength is from about 3 mm to about 300 mm.

132. The pharmaceutical composition of claim 131, wherein the specific wavelength is from about 50 mm to about 200 mm.

133. The pharmaceutical composition of claim 132, wherein the specific wavelength is from about 100 mm to about 150 mm.

134. The pharmaceutical composition according to any one of claims 100-133, wherein the API is a BCS Class II drug.

135. The pharmaceutical composition according to any one of claims 100-133, wherein the API is a BCS Class IV drug.

136. The pharmaceutical composition according to any one of claims 100-135, wherein the API is an API with a melting point of less than 250° C.

137. The pharmaceutical composition of claim 136, wherein the melting point is less than 200° C.

138. The pharmaceutical composition according to any one of claims 100-137, wherein the API is selected from anticancer agents, antiallergic agents, antifungal agents, psychiatric agents such as analgesics, consciousness level-altering agents such as anesthetic agents or hypnotics, nonsteroidal anti-inflammatory agents (NSAIDs), anthelmintics, antiacne agents, antianginal agents, antiarrhythmic agents, anti-asthma agents, antibacterial agents, anti-benign prostate hypertrophy agents, anticoagulants, antidepressants, antidiabetics, antiemetics, antiepileptics, antigout agents, antihypertensive agents, anti-inflammatory agents, antimalarials, antimigraine agents, antimuscarinic agents, antineoplastic agents, anti-obesity agents, antiosteoporosis agents, antiparkinsonian agents, antiproliferative agents, antiprotozoal agents, antithyroid agents, antitussive agent, anti-urinary incontinence agents, antiviral agents, anxiolytic agents, appetite suppressants, beta-blockers, cardiac inotropic agents, chemotherapeutic drugs, cognition enhancers, contraceptives, corticosteroids, Cox-2 inhibitors, diuretics, erectile dysfunction improvement agents, expectorants, gastrointestinal agents, histamine receptor antagonists, immunosuppressants, keratolytics, lipid regulating agents, leukotriene inhibitors, macrolides, muscle relaxants, neuroleptics, nutritional agents, opioid analgesics, protease inhibitors, and sedatives.

139. The pharmaceutical composition of claim 138, wherein the API is a chemotherapeutic drug, a psychiatric agent, an antiallergic agent, an antibiotic, an antihypertensive agent, an antifungal agent, an antiepileptic, or a nonsteroidal anti-inflammatory agent.

140. The pharmaceutical composition of claim 139, wherein the API is a chemotherapeutic drug.

141. The pharmaceutical composition of claim 139, wherein the API is an antibiotic.

142. The pharmaceutical composition of claim 139, wherein the API is a nonsteroidal anti-inflammatory agent.

143. The pharmaceutical composition of claim 142, wherein the API is ibuprofen or acetylsalicylic acid.

144. The pharmaceutical composition of claim 139, wherein the API is an antihypertensive agent.

145. The pharmaceutical composition of claim 144, wherein the API is nifedipine.

146. The pharmaceutical composition of claim 139, wherein the API is an antifungal agent.

147. The pharmaceutical composition of claim 146, wherein the API is indomethacin.

148. The pharmaceutical composition of claim 139, wherein the API is an antiepileptic.

149. The pharmaceutical composition of claim 148, wherein the API is carbamazepine.

150. The pharmaceutical composition of claim 139, wherein the API is a psychiatric agent.

151. The pharmaceutical composition of claim 150, wherein the API is aripiprazole.

152. The pharmaceutical composition of claim 139, wherein the API is an antiallergic agent.

153. The pharmaceutical composition of claim 152, wherein the API is tranilast.

154. The pharmaceutical composition according to any one of claims 100-153, wherein the co-former interacts with the API through one or more non-covalent interactions.

155. The pharmaceutical composition of claim 154, wherein the non-covalent interactions are ionic interactions, hydrogen bonding, halogen bonding, van der Waals forces, π-π interactions, or hydrophobic effects.

156. The pharmaceutical composition according to any one of claims 100-155, wherein the co-former and the active pharmaceutical ingredient interact with two or more non-covalent interactions.

157. The pharmaceutical composition according to any one of claims 100-155, wherein the co-former is a compound which modifies the solubility of the active pharmaceutical ingredient.

158. The pharmaceutical composition of claim 157, wherein the co-former is a compound which is sparingly soluble and modifies the solubility of the active pharmaceutical ingredient.

159. The pharmaceutical composition of claim 157, wherein the co-former is a compound which is sensitive to the environment and modifies the solubility of the active pharmaceutical ingredient.

160. The pharmaceutical composition of claim 159, wherein the compound is sensitive to the pH of the environment.

161. The pharmaceutical composition of claim 159, wherein the compound is sensitive to the temperature of the environment.

162. The pharmaceutical composition according to any one of claims 100-161, wherein the co-former is a compound that has no therapeutic effect.

163. The pharmaceutical composition according to any one of claims 100-161, wherein the co-former is a second active pharmaceutical ingredient.

164. The pharmaceutical composition of claim 163, wherein the second active pharmaceutical ingredient is for the same disease or disorder as the first active pharmaceutical ingredient.

165. The pharmaceutical composition of claim 163, wherein the second active pharmaceutical ingredient is for a different disease or disorder as the first active pharmaceutical ingredient.

166. The pharmaceutical composition according to any one of claims 100-165, wherein the co-former comprises one or more functional groups selected from amine, amide, a nitrogen containing heterocycle, carbonyl, carboxyl, hydroxyl, phenol, sulfone, sulfine, sulfinyl, sulfonyl, mercapto, and methyl thio.

167. The pharmaceutical composition of claim 166, wherein the functional group is a NH₂, OH, C(O), C(O)OH, SH, or a nitrogen containing heterocycle.

168. The pharmaceutical composition of claim 167, wherein the functional group is a nitrogen containing heterocycle, NH₂, OH, or SH.

169. The pharmaceutical composition according to any one of claims 100-168, wherein the co-former is a carboxylic acid.

170. The pharmaceutical composition of claim 169, wherein the co-former is malic acid.

171. The pharmaceutical composition according to any one of claims 100-168, wherein the co-former is a vitamin or a vitamin derivative.

172. The pharmaceutical composition of claim 171, wherein the co-former is nicotinamide.

173. The pharmaceutical composition according to any one of claims 100-168, wherein the co-former is a flavoring agent.

174. The pharmaceutical composition of claim 173, wherein the co-former is saccharin.

175. The pharmaceutical composition according to any one of claims 100-172, wherein the pKₐ of the active pharmaceutical ingredient and the pKₐ of the co-former have a pKₐ difference of less than 3.

176. The pharmaceutical composition of claim 175, wherein the pKₐ difference is less than 2.

177. The pharmaceutical composition of claim 176, wherein the pKₐ difference is less than 1.

178. The pharmaceutical composition of claim 177, wherein the pKₐ difference is less than 0.5.

179. The pharmaceutical composition according to any one of claims 100-178, wherein the pharmaceutical composition further comprises an excipient.

180. The pharmaceutical composition according to any one of claims 100-179, wherein the pharmaceutical composition is formulated for administration: orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularlly, intravitreally, liposomally, locally, mucosally, parenterally, rectally, subconjunctival, subcutaneously, sublingually, topically, transbuccally, transdermally, vaginally, in crèmes, in lipid compositions, via a catheter, via a lavage, via continuous infusion, via infusion, via inhalation, via injection, via local delivery, or via localized perfusion.

181. The pharmaceutical composition of claim 180, wherein the pharmaceutical composition has been formulated for oral administration.

182. The pharmaceutical composition of claim 181, wherein the pharmaceutical composition is present as a capsule, tablet, oral suspensions, oral films, or chewable dosages.

183. The pharmaceutical composition according to any one of claims 100-182, wherein the API is acetylsalicylic acid, indomethacin, ibuprofen, carbamazepine, or nifedipine and the co-former is nicotinamide or malic acid.

184. A pharmaceutical composition prepared according to the methods of any one of claims 1-99.

185. A method of treating or preventing a disease or disorder comprising administering a therapeutically effective amount of a pharmaceutical composition according to any one of claims 100-183 or a pharmaceutical composition prepared according to any one of claims 1-99, wherein the therapeutically active agent is useful for treating or preventing the disease or disorder.

186. A composition comprising:
(A) an active pharmaceutical ingredient (API);
(B) a co-former;
wherein at least 50% of the API and the co-former are present in a substantially liquid phase.

187. The composition of claim 186, wherein at least 80% of the API and the co-former are present in a substantially liquid phase.

188. The composition of claim 187, wherein at least 90% of the API and the co-former are present in a substantially liquid phase.

189. The composition of claim 188, wherein at least 95% of the API and the co-former are present in a substantially liquid phase.
